(19) United States
(12) Patent Application Publication
(10) Pub. No.: US 2015/0057325 A1
(43) Pub. Date: Feb. 26, 2015

(54) COMPOUNDS CAPABLE OF MODULATING/PRESERVING ENDOTHELIAL INTEGRITY FOR USE IN PREVENTION OR TREATMENT OF ACUTE TRAUMATIC COAGULOPATHY AND RESUSCITATED CARDIAC ARREST

(71) Applicant: Rigshospitalet, Kobenhavn O (DK)

(72) Inventors: Pär Johansson, Dosjebro (SE); Sisse Rye Ostrowski, Hellerup (DK)

(73) Assignee: Rigshospitalet, Kobenhavn O (DK)

(21) Appl. No.: 14/389,417

(22) PCT Filed: Mar. 26, 2013
(86) PCT No.: PCT/DK2013/050090

§ 371 Date: Sep. 30, 2014

(30) Foreign Application Priority Data
Mar. 30, 2012 (DK) PA 2012 70163

(51) Int. Cl. A61K 31/5585; A61K 45/06; G01N 33/68; A61K 31/5578

(52) U.S. Cl. CPC A61K 31/5585; A61K 31/5578; A61K 45/06; G01N 33/6893; G01N 2333/705; G01N 2800/224
USPC 514/401; 549/465; 514/469; 562/501; 514/573; 435/29; 435/7.21

(57) ABSTRACT

The present invention relates to novel uses of compounds that protect the endothelium, particularly prostacyclin and variants and derivatives thereof in the treatment or prevention of acute traumatic coagulopathy (ATC) and of patients resuscitating from cardiac arrest. The invention also relates to a method of identifying individuals at risk of developing ATC.

All patent and non-patent references cited in the application, or in the present application, are also hereby incorporated by reference in their entirety.

### FIELD OF INVENTION

The present invention relates to novel uses of compounds that protect the endothelium, particularly prostacyclin and variants and derivatives thereof in the treatment or prevention of acute traumatic coagulopathy (ATC) and of patients resuscitated from cardiac arrest. The invention also relates to a method of identifying individuals at risk of developing ATC at the scene of accident. In particular the present invention relates to treatment being initiated before the patient reaches the hospital, so-called pre-hospital treatment.

### BACKGROUND OF INVENTION

Worldwide, trauma continues to be a leading cause of death and disability, and in the industrialized countries accidents are the most frequent cause of death in persons younger than 40 years old [Peden et al 2002]. Coagulopathy plays a central role in trauma care and haemorrhage accounts for 40% of all trauma deaths [Sauaia et al 1995]. Bleeding control is extremely challenging in the presence of an established coagulopathy. The adverse outcomes of dysfunctional haemostasis are not limited to death from acute blood loss but also organ dysfunction or multiple organ failure is potential consequences of prolonged shock [Sauaia et al 1994; Sauaia et al 1995].

Coagulation is an integral part of inflammation and widespread activation of the coagulation system results in a systemic inflammatory response syndrome and increased susceptibility to sepsis [Moore et al 1996; Keel and Trentz 2005; Stahel et al 2007; Gando et al 2002; Ganter et al 2007; Maier et al 2007; Cohen et al 2010] further exacerbated by the immunologically adverse effects of blood transfusions. Database evaluations and clinical studies identify blood transfusion as an independent risk factor for adverse outcome in the critically ill patients [Malone et al 2003]. Coagulopathy also worsens outcomes from traumatic brain injury by an increased potential for intracranial haemorrhage and secondary neuronal loss [Allard et al 2009; Stein et al 1992].

Furthermore, acute traumatic coagulopathy (ATC) (also called acute coagulopathy of trauma shock (ACoTS), trauma induced coagulopathy (TIC), acute endogenous coagulopathy (AEC) of trauma, DIC with a fibrinolytic/hemorrhagic phenotype), herein called ATC, has recently been identified to be present in one of four trauma patients on admission and is associated with a 4-fold increase in mortality. ATC is characterized by hypocoagulation as evaluated by activated partial thromboplastin time (APTT), partial thromboplastin time (PTT), prothrombin time (PT) or thrombin time (TT) and increase in the natural anticoagulant activated protein C as well as an increased fibrinolytic activity as evaluated by D-dimer [Brohi et al 2003; MacLeod et al 2003; Maegele et al 2007; Brohi et al 2007; Brohi et al 2008; Wafaisade et al 2010]. The proposed drivers of ATC are tissue trauma and hypoperfusion, which results in the above mentioned plasmatic coagulation results.

It has previously been described that low dose prostacyclin in the hospital period is beneficial for outcome in patients with traumatic brain injury [Grande et al 2000; Naredi et al 2001], and several studies have reported that infusion of prostacyclin analogues reduces mortality and improves outcome in animals who have encountered a standardized trauma [Lefer et al 1979; Lefer and Araki 1983; Starling et al 1985; Levitt and Lefer 1986; Bitterman et al 1988a; Bitterman et al 1988b; Bitterman et al 1988c; Tamura 1992; Bentzer et al 2001; Bentzer et al 2003; Bentzer and Grande 2004; Lundblad et al 2008; Sahsivar et al 2009; Costantini et al 2009].

### SUMMARY OF INVENTION

The present invention relates to treatment and/or prevention of acute traumatic coagulopathy (ATC) and prevention of the sequelae following resuscitated cardiac arrest.

The inventors of the present invention have found that in patients with acute traumatic coagulopathy (ATC) the mortality is not affected by standard therapeutic approaches including blood transfusion therapy despite that retrospective reports indicate that high ratios of plasma and platelet concentrates to red blood cell concentrates improves outcome.

The inventors have also found that the high mortality associated with ATC is attributed to an acute systemic profound dysfunction of the endothelium, with degradation of the endothelial glycocalyx and ensuing shedding of natural endogenous anticoagulant molecules from the glycocalyx, resulting in hypocoagulability by TEG, prolonged activated partial thromboplastin time (APTT) and development of multiorgan failure in addition to the increased risk of bleeding due to combined effects of the trauma, hypoxia and disrupted vascular integrity.

As described above, ATC patients are at an increased risk of mortality and there thus exists a need for identifying patients with ATC or at risk of developing ATC.

Thus a first aspect of the present invention relates to a method for identifying ATC patients both in the hospital or other care unit and in a pre-hospital setting by use of different biomarkers and/or blood coagulation parameters.

A first embodiment of a first aspect of the invention relates to a method of diagnosing, measuring, monitoring or determining the likelihood of developing or actually having Acute Traumatic Coagulopathy, in a pre-hospital or hospital setting, wherein said method is capable of identifying a patient who has a significantly increased risk of developing Acute Traumatic Coagulopathy, said method comprising the steps of:
i. determining and/or measuring the concentration of at least one of Syndecan-1, B-glucose, B-lactate or APTT in a whole blood sample from the patient,
ii. comparing said concentration with a predetermined cutoff value, wherein said cutoff value is:
   a) Syndecan-1 2-fold higher than normal and/or
   b) B-glucose 50% higher than normal and/or
   c) B-lactate 3.5 fold higher than normal and/or
   d) APTT above normal,
      wherein a Syndecan-1 value higher than the cutoff value and/or a B-glucose value higher than the cutoff value and/or a B-lactate value higher than the cutoff and/or a APTT value higher than the cutoff value is indicative of a significantly increased risk of developing or having Acute Traumatic Coagulopathy.

In particularly, individuals sustaining trauma having one or more of the values higher than the cutoff have evidence of profound endothelial cell and endothelial glycocalyx damage and/or degradation, and hence ATC, or a significantly increased risk of developing ATC as compared to individuals not having any of the values higher than the cutoff.

Determination of Syndecan-1, B-glucose, B-lactate and APTT can be carried out at the place of the trauma, i.e. pre-hospital, or en route to the hospital and accordingly, a treatment can be initiated even before the patient has reached the hospital.

Another embodiment of the first aspect relates to a method of diagnosing, measuring, monitoring or determining the likelihood of developing Acute Traumatic Coagulopathy, wherein said method is capable of identifying patients who have acquired or have a significantly increased risk of developing Acute Traumatic Coagulopathy, said method comprising the steps of:
i. determining and/or measuring at least one of the viscoelastical data points R, Angle and MA by thromboelastography (TEG) in a whole blood sample from the patient, such as in a citrated whole blood sample, such as in a citrated whole blood sample activated by kaolin,
ii. comparing said concentration with a predetermined cutoff value, said cutoff value being an equivalent to a cutoff value determined by TEG in a citrated whole blood sample activated by kaolin wherein said cutoff value is:
   a) R higher than 8.0 minutes, such as higher than 11 minutes, such as higher than 12 minutes and/or
   b) Angle lower than 60°, such as lower than 55° and/or,
   c) MA lower than 51 mm, such as lower than 50 mm and/or
   d) Ly30 higher than 7% such as higher than 8%,
      wherein an R-value higher than the cutoff value and/or an Angle-value lower than the cutoff value and/or a MA lower than the cutoff value and/or a Ly30 value higher than the cutoff value is indicative of a significantly increased risk of developing Acute Traumatic Coagulopathy as compared to a human being wherein neither R or Ly30 are higher or Angle-value or MA are lower than the cutoff value.

Another embodiment of the first aspect relates to a method of diagnosing, measuring, monitoring or determining the likelihood of developing Acute Traumatic Coagulopathy, wherein said method is capable of identifying patients who already have ATC or have a significantly increased risk of developing Acute Traumatic Coagulopathy, said method comprising the steps of
i) determining and/or measuring at least one of the viscoelastical data points Clotting time, Clot formation time, Angle, CA5 and MCF by thromboelastometry (ROTEM) in a whole blood sample from the patient, such as in a citrated whole blood sample, such as in a citrated whole blood sample activated by kaolin,
ii) comparing said concentration with a predetermined cutoff value, said cutoff value being an equivalent to a cutoff value determined by TEG in a citrated whole blood sample activated by kaolin wherein said cutoff value is:
   a) Clotting time higher than 65 seconds, such as higher than 70 seconds and/or
   b) Clot formation time higher than 110 seconds, such as higher than 120 seconds and/or
   c) Angle lower than 75 degrees, such as lower than 70 degrees and/or
   d) CA5 lower than 45 mm, such as lower than 40 mm and/or,
   e) MCF lower than 60 mm, such as lower than 55 mm and/or,
      wherein a clotting time higher than the cutoff value and/or a clot formation time higher than the cutoff value, an Angle-value lower than the cutoff value and/or a CA5 value lower than the cutoff value and/or a MCF lower than the cutoff value is indicative of a significantly increased risk of developing organ failure including MOF as compared to a human being wherein neither clotting time or clot formation time are higher than the cutoff value or Angle, CA5 or MCF values are lower than the cutoff value.

Furthermore, the invention relates to a diagnostic kit for diagnosing individuals at risk of developing or having Acute Traumatic Coagulopathy. In a preferred embodiment the diagnostic kit includes means for determining Syndecan-1, or B-glucose or B-lactate or APTT simultaneously, separately or sequentially, more preferably means for determining Syndecan-1, and/or B-glucose, most preferably means for determining Syndecan-1.

The inventors have found that a prostacyclin compound, such as prostacyclin (PGI2), and prostacyclin (PGX), thereof may be useful in the treatment and prevention of ATC.

The prostacyclin compound may be any suitable prostacyclin compound, such as iloprost, flolan, beraprost or Epoprostenol. Furthermore, the prostacyclin compound may be a prostacyclin variant or analogue.

Also, the prostacyclin compound may be administered in combination with any one of another compound capable of modulating and/or preserving the endothelial integrity, such as nitrogen oxide, glycocorticoids, antithrombin, activated protein C (APC), insulin, N-acetylcysteine, albumin, valproate, plasma, oxygen carriers or variants thereof.

In yet another embodiment the prostacyclin compound may be administered in combination with antagonists of adrenergic receptors.

In yet another embodiment the prostacyclin compound may be administered in combination with agonists of adrenergic receptors.

Thus, one object of the present invention relates to a compound as described above used in prevention or treatment of Acute Traumatic Coagulopathy whereas another aspect relates to a compound as described above for use in treatment of patients resuscitated from cardiac arrest, in particularly the sequelae from cardiac arrest.

Thus an object of the present invention relates to a method of treating or preventing a disease selected from the group consisting of Acute Traumatic Coagulopathy and cardiac arrest comprising administering one or more compounds as described above.

Another object of the present invention relates to the use of one or more compounds as described above in the manufacture of a medicament for the treatment or prevention of a disease selected from the group consisting of Acute Traumatic Coagulopathy and sequelae from cardiac arrest.

A further aspect relates to a kit for use in the treatment and/or prophylaxis of a disease selected from the group consisting of Acute Traumatic Coagulopathy and cardiac arrest comprising
i) a prostacyclin compound as described above,
ii) optionally an aqueous medium to dissolve the compound, and
iii) optionally instructions for use.

A further aspect relates to a kit for use in the treatment and/or prophylaxis of a disease selected from the group consisting of Acute Traumatic Coagulopathy and cardiac arrest according to any of the preceding claims, comprising
i) a prostacyclin compound as described above,
ii) optionally another compound which is any one or more of:
   a. capable of modulating and/or preserving the endothelial integrity and/or
   b. an antagonist of adrenergic receptors or
   c. an agonist of adrenergic receptors, for simultaneous, separate or sequential administration,
iii) optionally an aqueous medium to dissolve the compound, and
iv) optionally instructions for use.

Yet another aspect relates to a method for the treatment or prophylaxis of a disease selected from the group consisting of Acute Traumatic Coagulopathy and cardiac arrest of a subject in need of such a treatment, the method comprises administration of an effective dose of compound as described above.

Another object of the present invention relates to a pharmaceutical composition comprising a compound as described above for the treatment or prophylaxis of a disease selected from the group consisting of Acute Traumatic Coagulopathy and resuscitated cardiac arrest.

Additional aspects of the present invention and particular embodiments will be apparent from the description below as well from the appended claims.

### DESCRIPTION OF FIGURES

FIG. 1 shows the TEG assay, setup as well as result.

FIG. 2 shows the Multiple Platelet function Analyzer (Multiplate) as well as the result.

FIG. 3 shows the measured TEG values.

FIG. 4 shows the measured Multiplate values.

FIG. 5 shows Mortality (5A), Injury Severity Score (ISS) (5B), Adrenaline concentration (5C), and Noradrenaline concentration (5D) in individuals having High and Low Glycocalyx degradation, respectively.

FIG. 6 shows the correlation between Syndecan-1 values and adrenaline.

FIG. 7 shows the principle of TEG and ROTEM. The following parameters are derived from a TEG tracing; R, the time from start of analysis until initial clot formation (at 2 mm amplitude); Angle, representing velocity of clot formation; MA, maximal amplitude, the maximal physical clot strength; Lysis AUC, the area under the fibrinolysis curve calculated from MA. The values in FIG. 7 reflects TEG Ly30>8% and ROTEM CL>8% hyperfibrinolysis.

FIG. 8: A: Levels of thrombomodulin over time during and following prostacyclin administration in healthy individuals. B: Levels of Protein C over time during and following prostacyclin administration in healthy individuals.

FIG. 9: A: B: Levels of PAI-1 over time during and following prostacyclin administration in healthy individuals. B: Levels of Antithrombin over time during and following prostacyclin administration in healthy individuals.

FIG. 10: A: B: Levels of Histone-complexed DNA over time during and following prostacyclin administration in healthy individuals. B: Levels of HMGB1 over time during and following prostacyclin administration in healthy individuals.

FIG. 11: A: B: Levels of Syndecan-1 over time during and following prostacyclin administration in healthy individuals. B: Levels of TFPI over time during and following prostacyclin administration in healthy individuals.

### DEFINITIONS

Acute traumatic coagulopathy (ATC) (other names acute coagulopathy of trauma shock (ACoTS), trauma induced coagulopathy (TIC), acute endogenous coagulopathy (AEC) of trauma, DIC with a fibrinolytic/hemorrhagic phenotype, but herein called ATC) may be defined as an impairment of hemostasis that may occur early after injury and is associated with a four-fold higher mortality, increased transfusion requirements and increased risk of developing or having organ failure.

The terms prothrombin time (PT) and its derived measures of prothrombin ratio (PTr or PR) and international normalized ratio (INR) as used herein are intended to mean measures of the extrinsic pathway of coagulation. They are used to determine the clotting tendency of blood. The reference range for prothrombin time is usually around 12-15 seconds; the normal range for the INR is 0.8-1.2. PT measures factors I, II, V, VII, and X. It may be used in conjunction with the activated partial thromboplastin time (APTT) which measures the intrinsic pathway. The normal value for APTT is from 23-35 seconds.

The term “International Sensitivity Index” (ISI) as used herein is intended to mean how a particular batch of tissue factor compares to an internationally standardized sample (ISI is assigned by the manufacturer of said tissue factor). The ISI is usually between 1.0 and 2.0.

The term “International normalized ratio” as used herein is intended to mean a standardized ratio of a patient's prothrombin time to a normal (control) sample, raised to the power of the ISI value for the analytical system used:

$$INR = {( \frac{{PT}_{test}}{{PT}_{normal}} )}^{ISI}$$

The result (in seconds) for a prothrombin time performed on a normal individual will vary depending on what type of analytical system it is performed. This is due to the differences between different batches of manufacturer's tissue factor used in the reagent to perform the test.

The term “modulating and/or preserving endothelial integrity” is intended to mean pharmacological treatment aiming at maintaining the endothelium in a quiescent inactivated, anti-adhesive and anti-coagulant state. Thus a “compound capable of modulating/preserving endothelial integrity” is intended to mean any compound that may assist in maintaining the endothelium in a quiescent inactivated anti-coagulant and anti-adhesive state and/or may assist in inducing the endothelium into such a quiescent inactivated anti-coagulant and anti-adhesive state.

The term “Endothelial modulators” encompasses any agent that affects the endothelium to either maintain or develop into a state which optimally preserves and ensures vascular integrity. In a state with vascular integrity, the endothelium exerts anti-adhesive, anti-thrombotic and anti-inflammatory properties.

The term “hypercoagulability” used herein will reflect an increased coagulation activity in the initiation phase (decreased R), and/or increased thrombin burst (increased Angle) and/or increased clot strength (increased MA) as evaluated by TEG as compared to the normal reference.

The term “hypocoagulability” used herein will reflect decreased coagulation activity in the initiation phase (increased R), and/or increased thrombin burst (decreased Angle) and/or increased clot strength (decreased MA) as evaluated by TEG as compared to the normal reference.

Hypocoagulability refers to a coagulopathy where the normal haemostatic process is impaired resulting in delayed initiation of coagulation activation, reduced coagulation amplification and propagation resulting in reduced or absent clot formation.

Hypocoagulability can also be due to abnormally increased fibrinolytic activity resulting in decreased clot stability due to increased rate of clot breakdown as depicted by an increased lysis by TEG (>8% 30 min after MA is reached). These two forms of hypocoagulability can exist together simultaneously or alone, i.e. independent of each other.

The first type of hypocoagulability can be identified by an APTT score above 35 sec. and/or PT above 1.2 and/or PTr above 1.2 and/or fibrinogen below 1.0 g/L and/or platelet count below 100×10E9/l.

The second type of hypocoagulability can be identified by the prevalence of increased D-dimer such as D-dimer being increased 5-10 fold above normal and an increased value of tPA such as a value increased 2-3 fold above normal.

The term “homeostasis” refers to the body's ability to regulate physiologically its inner environment to ensure its stability. An inability to maintain homeostasis may lead to death or a disease.

The term “shock” is used in the conventional clinical meaning, i.e. shock is a medical emergency in which the organs and tissues of the body are not receiving an adequate flow of blood. This deprives the organs and tissues of oxygen (carried in the blood) and allows the build-up of waste products. Shock is caused by four major categories of problems: cardiogenic (meaning problems associated with the heart's functioning); hypovolemic/haemorrhagic (meaning that the total volume of blood available to circulate is low); neurogenic (caused by severe injury to the central nervous system) and septic (caused by overwhelming infection, usually by bacteria).

A “subject” includes humans and other mammals, and thus the methods are applicable to both human therapy and veterinary applications, in particular to human therapy. The term “mammal” includes humans, non-human primates (e.g. baboons, orangutans, monkeys), mice, pigs, cows, goats, cats, dogs, rabbits, rats, guinea pigs, hamsters, horse, monkeys, sheep or other non-human mammals.

“Treatment”, as used in this application, is intended to include treatment of acute traumatic coagulopathy (ATC) and treatment of the sequelae of resuscitated cardiac arrest. Prevention is intended to mean treatment in order to reduce risk of ATC and of sequelae of resuscitated cardiac arrest.

“Trauma” as used herein is intended to mean any body wound or shock produced by sudden physical injury, as from accident, injury, or impact to living tissue caused by an extrinsic agent i.e. injury to living tissue caused by an extrinsic agent, examples are blast trauma, blunt trauma, penetrating trauma, trauma caused by chemical injury (spills, warfare or intoxication), radiation or burns.

With variant and analogue is meant any variant and analogue of a compound capable of modulating and/or preserving endothelial integrity, particularly variants and/or analogues of prostacyclin which are functional equivalents of said compound.

As used herein, “dose” shall mean a dose sufficient to produce the desired effect in relation to the conditions for which it is administered, in particular an amount of a compound capable of modulating/preserving endothelial integrity that is effective to stop, reduce or prevent the coagulopathy or cardiac arrest shall be described as the “effective dose”, “therapeutically effective dose” or “effective amount”. Normally the dose should be capable of preventing or lessening the severity or spread of the condition or indication being treated. The exact dose will depend on the circumstances, such as the condition being treated, the administration schedule, whether the compound capable of modulating/preserving endothelial integrity is administered alone or in conjunction with another therapeutic agent or compound capable of modulating/preserving endothelial integrity, the plasma half-life of the compound capable of modulating/preserving endothelial integrity and the general health of the subject.

### DETAILED DESCRIPTION OF THE INVENTION

As described herein above the inventors have found that in patients with acute traumatic coagulopathy (ATC) the mortality is not affected by standard therapeutic approaches to revert or treat coagulopathy including blood transfusion therapy. Instead the inventors have found that endothelial dysfunction may be part of the pathogenesis of ATC.

The vascular endothelium comprises a single layer of cells (endothelial cells) that lines each and every vessel in the body, covering a total surface area of 4-7000 m2 and having a total weight of 1 kg. Healthy endothelial cells contribute to 1) prevent thrombosis formation, 2) exchange fluid/macromolecules across blood and tissue (trans-/paracellular), 3) control blood flow, 4) quiescence of the inflammatory response and 5) immune surveillance. On top of a healthy endothelium lies the endothelial glycocalyx, a 0.2-1 μm thick, negatively charged carbohydrate-rich layer that contributes to the vasculo-protective effects of the vessel wall and contributes to the maintenance of vascular integrity. The glycocalyx is connected to the endothelium through several “backbone” molecules (e.g., proteoglycans like syndecan-1, glycoproteins and various endothelial adhesion molecules, integrins and components of the coagulation and fibrinolytic systems). These molecules form a network in which soluble molecules, either plasma- or endothelium-derived, are incorporated.

Within the glycocalyx lies a fixed non-circulating plasma volume (also called the endothelial surface layer) with a total volume of 1 litre in adults, thus representing one third of the total plasma volume. The large dimension of the endothelial glycocalyx reveals a big and very important compartment of the circulation. The glycocalyx constituents including plasma and plasma proteins are in dynamic equilibrium with the flowing plasma, and upon damage to the glycocalyx, a substantial part of the absorbed layer of plasma components and the glycocalyx are dissolved into the flowing blood.

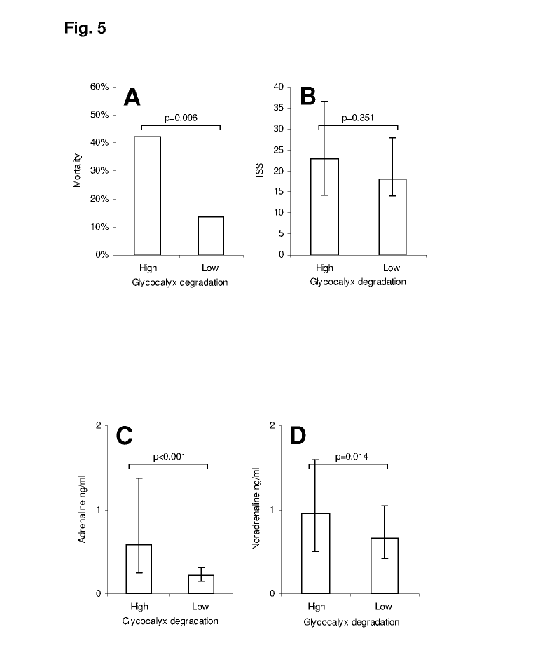

The inventors have found that the degree of endothelial glycocalyx dysfunction/damage/degradation (as evaluated by Syndecan-1, the protein backbone of the glycocalyx) correlates with adrenaline concentration in trauma patients, independent on injury severity, indicating that an important cause of acute traumatic coagulopathy is the catecholamine induced destruction of the endothelial glycocalyx (FIG. 5). It has also been found, that in patients with the same degree of tissue injury as evaluated by the injury severity score (ISS), the degree of glycocalyx damage, as evaluated by Syndecan-1, determines outcome of the patients. Patients responding to trauma by high Syndecan-1 shedding/degradation have a threefold increase in mortality as compared to patients with the same degree of trauma but responding with a low Syndecan-1 shedding/degradation (FIG. 5B). Thus, the patient's response to the trauma, with either high or low glycocalyx shedding/degradation, rather than the absolute injury severity, determines the patients risk of dying.

Patients with a high degree of shedding/degradation also had significantly increased adrenaline and noradrenaline as compared to patients with low level glycocalyx shedding/degradation, further emphasizing the mechanistic link between catecholamines and glycocalyx shedding/degradation. It follows that the degree of damage to the endothelium, i.e. the endothelial cells and the glycocalyx, determines the patients risk of having acquired ATC.

The present inventors have further found that a compound as described above, and in particular prostacyclin or a variant or analogue thereof, may be useful in the treatment and prevention of ATC as well as sequelae from cardiac arrest.

### Prostacyclin Compounds

In particularly, the invention relates to the treatment using prostacyclin or a variant thereof. Prostacyclin, a metabolite of arachidonic acid, is a naturally occurring prostaglandin with potent vasodilatory activity and inhibitory activity of platelet aggregation, released by healthy endothelial cells. Prostacyclin performs its function through a paracrine signalling cascade that involves G protein-coupled receptors on nearby platelets and endothelial cells.

In one embodiment the prostacyclin variant is selected from the group consisting of beraprost sodium, epoprostenol sodium (flolan), iloprost, iloprost in combination with bosentan, iloprost in combination with sildenafil citrate, treprostinil, pegylated treprostinil, treprostinil diethanolamine and treprostinil sodium. Further compounds are 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, and 15-deoxy-16[alpha]-hydroxy-16[beta],20-dimethyl-[DELTA]6,6a-6a-carba PGI1, pentoxifylline (1-{5-oxohexyl}-3,7-dimethylxanthine).

The modulating/preserving effect on endothelial integrity is mediated by binding of the prostacyclin compound to endothelial prostacyclin receptors with ultimate rise in cytosolic cAMP and Protein Kinase A activation. This leads to smooth muscle relaxation and vasodilatation with improved microvascular perfusion and “cytoprotection” through stabilization of lysozomal and cell membranes with reduced inflammation.

In a preferred embodiment the prostacyclin compound has a half time of less than 4 hours (such as Treprostinil), preferably less than 1 hours (such as Beraprost (35-40 min)), more preferably less than ½ hour (such as Iloprost (20-30 min)), preferably less than 5 min (such as Epoprostenol (0.5-3 min)).

The prostacyclin compound is in particular prostacyclin PGI2, prostacyclin PGX, prostacyclin (Epoprostenol) or variants thereof, such as beraprost sodium, epoprostenol sodium, iloprost, iloprost in combination with bosentan, iloprost in combination with sildenafil citrate, treprostinil, pegylated treprostinil, treprostinil diethanolamine and treprostinil sodium. Further compounds are 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyacetic acid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, and 15-deoxy-16[alpha]-hydroxy-16[beta],20-dimethyl-[DELTA]6,6a-6a-carba PGI1, pentoxifylline (1-{5-oxohexyl}-3,7-dimethylxanthine).

Trade names for prostacyclins include, but are not limited to: flolan, remodulin, Iloprost (Ilomedin) and ventavis.

### Combination Treatment

The compounds to be applied in the method of the present invention may be administered with at least one other compound. The compounds may be administered simultaneously, either as separate formulations or combined in a unit dosage form, or administered sequentially. It is thus also contemplated that one compound may be administered intravenously for example in combination with another compound that is administered orally.

### Agents Modulating/Preserving Endothelial Integrity

The prostacyclin compound may be combined with agents capable of modulating and/or preserving endothelial integrity and/or a variety of other compounds in the treatment or prevention of ATC and/or sequelae from cardiac arrest.

The endothelium maintains under physiological conditions a normal vascular function by regulating the balance between vasodilator and vasoconstrictor mediators and by regulating the expression of adhesion receptors. Endothelial modulators encompass any agent that affects the endothelium to either maintain or develop into a non-activated quiescent state, which optimally preserves and ensures vascular integrity. In a state with vascular integrity, the endothelium exerts anti-inflammatory and anti-thrombotic properties down-regulating and counteracting platelet activation through the generation of PGI2 (prostaglandin 12, prostacyclin) and through the production of ADPase, the latter catalyzing the degradation of ADP. Endothelial cells can also prevent the activation of the coagulation cascade by expressing surface molecules with anticoagulant properties such as heparan sulfate, dermatan sulphate (both constituents of the endothelial glycocalyx, residing on a backbone of the Syndecan-1 protein), tissue factor pathway inhibitor (TFPI), protein S (PS) and thrombomodulin (TM). Endothelial cells express plasminogen, tissue-type plasminogen activator (tPA), urokinase-type plasminogen activator (uPA), urokinase-type plasminogen activator receptor (uPAR) as well as membrane-associated plasminogen activator binding sites, thus favouring the generation of plasmin, and they express endothelial protein C receptor (EPCR), which enhances the anticoagulant activity. It follows that any of these naturally occurring compounds may be used as markers of endothelial damage.

The endothelial modulators may be selected from any of the classes of compounds (1-10) described below:
1. Compounds with modulating/preserving endothelial effects such as nitric oxide (also Endothelium Derived Relaxing Factor) produced by healthy endothelial cells induce vasodilatation and favours an anti-adhesive and anti-inflammatory phenotype of the endothelium through a rise in cytosolic cGMP [Cines et al 1998; Zardi et al 2005].
2. Clinical drugs involved in redox control of endothelial functions such as: HMG-CoA reductase inhibitors (Fluvastatin, Lovastatin, Pravastatin, Simvastatin), Angiotensin-receptor antagonists and ACE inhibitors (Captopril, Zofenopril, Enalapril, Ramipril, Quinapril, Perindopril, Lisinopril, Benazepril, Fosinopril, Casokinins, lactokinins), Peroxisome proliferator-activated receptors (PPARs), NADPH oxidase, Xanthine oxidase, PETN, Heparan sulfates (PI-88), heparan sulfate mimetics, Activators of oxidized/heme-free sGC (BAY 58-2667), and Anti-PECAM/SOD.
3. Compounds that directly modulate endothelial barrier function through modulating effects on sphingosine-1-phosphate (S1P)-receptors (eg.: FTY720, AA-R, AAL-S, KRP-203, AUY954, CYM-5442, SEW2871, W146, W140, VPC44116, VPC23019, JTE-013) [Marsolais et al 2009].
4. Antibodies and/or other molecules including activated protein C against/antagonizing histones that through their inhibition diminishes histone-mediated endothelial damage and/or microthrombi formation and/or fibrin deposition [Xu et al 2009].
5. Compounds enhancing the natural anticoagulant pathways and hence protecting the endothelium such as but not exclusively: Protein C pathway (Activated protein C (APC, Drotrecogin alfa, Xigris), protein C, compounds that either mimics and/or protects from degradation and/or enhances soluble thrombomodulin and/or EPCR and/or protein S), Antithrombin III (ATIII) (or ATIII like compounds and/or compounds that enhance ATIII function) and tissue factor pathway inhibitor (TFPI) (or TFPI compounds and/or compounds that enhance TFPI function).
6. Glucocorticoids
7. Insulin
8. N-acetylcysteine
9. Albumin
10. Hemoglobin based oxygen carriers
11. Human plasma such as Fresh Frozen Plasma (FFP), lyophilized plasma, and non-frozen human plasma (FP-24).
12. Valproate

Thus it is an object of the present invention to administer prostacyclin or variants or analogues hereof in combination with any of the above mentioned compounds for the treatment of ATC or cardiac arrest sequelae: preferably, prostacyclin is administered in combination with compounds enhancing the natural anticoagulant pathways such as APC, thrombomodulin and/or antithrombin.

A further object of the present invention is the administration of prostacyclin or variants or analogues hereof in combination with Human plasma, such as Fresh Frozen Plasma (FFP) or lyophilized plasma or Human non-frozen Plasma and/or valproate for the treatment of ATC or cardiac arrest sequelae.

Another object of the present invention is the administration of prostacyclin or variants or analogues hereof in combination with any of the above mentioned compounds for the treatment of ATC or cardiac arrest sequelae; preferably, prostacyclin is administered in combination with compounds with modulating/preserving endothelial effects such as nitric oxide.

Another object of the present invention is the administration of prostacyclin or variants or analogues hereof in combination with any of the above mentioned compounds for the treatment of ATC or cardiac arrest sequelae; preferably, prostacyclin is administered in combination with Glucocorticoids, Insulin, N-acetylcysteine, Albumin and/or Hemoglobin based oxygen carriers.

A further object of the present invention is the administration of prostacyclin or variants or analogues hereof in combination with any of the above mentioned compounds for the treatment of ATC or cardiac arrest sequelae; preferably, prostacyclin is administered in combination with drugs involved in redox control of endothelial functions such as: HMG-CoA reductase inhibitors (Fluvastatin, Lovastatin, Pravastatin, Simvastatin), Angiotensin-receptor antagonists and ACE inhibitors (Captopril, Zofenopril, Enalapril, Ramipril, Quinapril, Perindopril, Lisinopril, Benazepril, Fosinopril, Casokinins, lactokinins), Peroxisome proliferator-activated receptors (PPARs), NADPH oxidase, Xanthine oxidase, PETN, Heparan sulfates (PI-88), heparan sulfate mimetics, Activators of oxidized/heme-free sGC (BAY 58-2667), and/or Anti-PECAM/SOD.

A further object of the present invention is the administration of prostacyclin or variants or analogues hereof in combination with any of the above mentioned compounds for the treatment of ATC or cardiac arrest sequelae; preferably, prostacyclin is administered in combination with compounds that directly modulate endothelial barrier function through modulating effects on sphingosine-1-phosphate (S1P)-receptors such as FTY720, AA-R, AAL-S, KRP-203, AUY954, CYM-5442, SEW2871, W146, W140, VPC44116, VPC23019, and/or JTE-013).

### Treatment Using Antagonist of Adrenergic Receptors

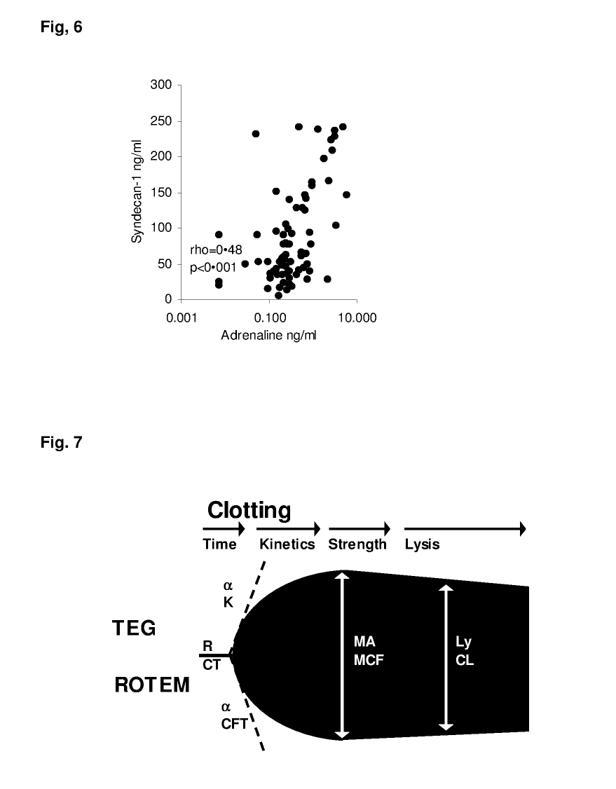

The inventors have found that the degree of endothelial damage/disruption correlates to the level of circulating adrenalin (FIG. 6) and since endothelial damage/disruption as evaluated by Syndecan-1 correlates with mortality in trauma patients an intervention aiming at modulating the sympathoadrenal response may be beneficial in these patients.

This is further supported by retrospective investigations of trauma patients reporting that those who were on adrenergic beta-blocker therapy demonstrated improved survival compared to patients not taking beta-blockers [Arbabi et al 2007]. Furthermore, in an in vitro study Rough et al. performed In vitro studies in RAW 264.7 cells using epinephrine (50 mmol/L) with or without a2- and b2-receptor blockade demonstrating that b2-receptor blockade reduces macrophage cytokine production and improves survival showing the critical importance of catecholamines to the immunologic response in surgery [Rough et al 2009].

Therefore, in one embodiment the endothelial modulator, such as prostacyclin, is administered in combination with modulators of the effect of the sympathoadrenal transmittor adrenalin. The compounds of the combination may be administered simultaneously, separate, or sequentially. Also, the prostacyclin compound may be administered together with one or more endothelial modulating compounds and one or more agonists or antagonists of adrenergic receptors.

In the following adrenergic receptor modulators to be co-administered with the endothelial modulator are listed:

### Alpha-1 (α₁) Adrenergic Receptor Agonists

Methoxamine
Methylnorepinephrine
Oxymetazoline
Phenylephrine

### Alpha-2 (α₂) Adrenergic Receptor Agonists

Clonidine
Guanfacine
Guanabenz
Guanoxabenz
Guanethidine
Xylazine
Methyldopa
Fadolmidine

### Undetermined α Adrenergic Receptor Agonists

amidephrine
amitraz
anisodamine
apraclonidine
brimonidine
cirazoline
detomidine
dexmedetomidine
epinephrine
ergotamine
etilefrine
indanidine
lofexidine
medetomidine
mephentermine
metaraminol
methoxamine
midodrine
mivazerol
naphazoline
norepinephrine
norfenefrine
octopamine
oxymetazoline
phenylpropanolamine
rilmenidine
romifidine
synephrine
talipexole
tizanidine

### Beta-1 Adrenergic Receptor Agonists

Dobutamine
Isoproterenol
Xamoterol
epinephrine

### Beta-2 Adrenergic Receptor Agonists

salbutamol
Fenoterol
Formoterol
Isoproterenol
Metaproterenol
Salmeterol
Terbutaline
Clenbuterol
Isoetarine
pirbuterol
procaterol
ritodrine
epinephrine

### Undetermined Beta Adrenergic Receptor Agonists

arbutamine
befunolol
bromoacetylalprenololmenthane
broxaterol
cimaterol
cirazoline
denopamine
dopexamine
etilefrine
hexoprenaline
higenamine
isoxsuprine
mabuterol
methoxyphenamine
nylidrin
oxyfedrine
prenalterol
ractopamine
reproterol
rimiterol
tretoquinol
tulobuterol
zilpaterol
zinterol

### Alpha-1 (α₁) Adrenergic Receptor Antagonists

Alfuzosin
Arotinolol
Carvedilol
Doxazosin
Indoramin
Labetalol
Moxisylyte
Phenoxybenzamine
Phentolamine
Prazosin
Silodosin
Tamsulosin
Terazosin
Tolazoline
Trimazosin

### Alpha-2 (α₂) Adrenergic Receptor Antagonists

Atipamezole
Cirazoline
Efaroxan
Idazoxan
Mianserin
Mirtazapine
Napitane
Phenoxybenzamine
Phentolamine
Rauwolscine
Setiptiline
Tolazoline
Yohimbine

### Beta-1 Adrenergic Receptor Antagonists

Acebutolol
Atenolol
Betaxolol
Bisoprolol
Esmolol
Metoprolol
Nebivolol

### Beta-2 Adrenergic Receptor Antagonists

Butaxamine
ICI-118,551

### Non-Selective Beta-Blockers

Bucindolol
Alprenolol
Carteolol
Carvedilol (has additional α-blocking activity)
Labetalol (has additional α-blocking activity)
Nadolol
Penbutolol
Pindolol
Propranolol
Sotalol
Timolol

### Beta-3 Adrenergic Receptor Antagonists

SR 59230A (has additional α-blocking activity)
   Other Modulators of the Sympathoadrenal System that can be Combined with Prostacyclin.
Levosimendan
Hydrocortizone
Arginine vasopressin

An object of the present invention is thus the administration of prostacyclin or variants or analogues hereof in combination with any of the above mentioned compounds for the treatment of ATC or cardiac arrest sequelae; preferably, prostacyclin is administered in combination with adrenergic receptor agonists such as, but not limited to: phenylephrine, Clonidine and/or epinephrine.

Another object of the present invention is thus the administration of prostacyclin or variants or analogues hereof in combination with any of the above mentioned compounds for the treatment of ATC or cardiac arrest sequelae; preferably, prostacyclin is administered in combination with beta receptor agonists such as, but not limited to: Dobutamine, Isoproteterenol and/or epinephrine.

Another object of the present invention is thus the administration of prostacyclin or variants or analogues hereof in combination with any of the above mentioned compounds for the treatment of ATC or cardiac arrest sequelae; preferably, prostacyclin is administered in combination with alpha and/or beta receptor antagonists and/or any of the above mentioned beta-blockers

### Dosages

As used herein, “dose” shall mean any concentration of the compounds administered to the patient resulting in maintaining the endothelium in a quiescent state. A dose sufficient to produce the desired effect in relation to the conditions for which it is administered shall be described as the “effective dose” or “effective amount”.

As will be understood by the person skilled in the art, amounts effective for this purpose will depend on the number and functionality of endothelial cells in the patient and the number of receptors on the respective endothelial cells.

The dosage requirements will vary with the particular drug composition employed, the route of administration and the particular subject being treated. Ideally, a patient to be treated by the present method will receive a pharmaceutically effective amount of the compound in the maximum tolerated dose, generally no higher than that required before drug resistance develops.

Administration of the compounds and/or compositions of the present invention are to be given to a subject resulting in a systemic concentration of the compounds. Methods of administration include enteral, such as oral, sublingual, gastric or rectal and/or parenterally, that is by intravenous, intraarterial, intramuscular, subcutaneous, intranasal, intrapulmonary, intrarectal, intraosseous, intravaginal or intraperitoneal administration. The intramuscular, sublingual, subcutaneous and intravenous forms of parenteral administration are generally preferred. Appropriate dosage forms for such administration may be prepared by conventional techniques. The compounds may also be administered by inhalation that is by intranasal and oral inhalation administration. Appropriate dosage forms for such administration, such as an aerosol formulation or a metered dose inhaler, may be prepared by conventional techniques.

As will be understood by the person skilled in the art, amounts effective for this purpose will depend on the severity of the disease or injury as well as the weight and general state of the subject. The dose is preferably given by the parenteral administration route, notably the intravenous, intramuscular, intraosseous and/or the subcutaneous, sublingual, trans-mucosal, intrapulmonal and intra-alveolar route.

The compounds according to the invention may be administered with at least one other compound. The compounds may be administered simultaneously, either as separate formulations or combined in a unit dosage form, or administered sequentially.

Normally the dose should be capable of preventing or lessening the severity or spread of the condition or indication being treated. The exact dose will depend on the circumstances, such as the condition being treated, the administration schedule, whether the compounds are administered alone or in conjunction with another therapeutic agent, the plasma half-life of the compounds and the general health of the subject.

The dosages given in the following is contemplated to be in the same order of magnitude irrespective of the parenteral administration route.

The term “unit dosage form” as used herein refers to physically discrete units suitable as unitary dosages for human and animal subjects, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host

In a specific embodiment the compound capable of modulating/preserving endothelial integrity particularly prostacyclin (PGI2), prostacyclin (PGX), or variants thereof, most preferably iloprost or flolan, the dose administered will for parenteral routes, in particular intravenous, intramuscular, and/or subcutaneous routes, in a single or repeated bolus dose corresponding to maintaining a systemic concentration of about 0.5-4.0 ng/kg for a period of time, such as for 10 minutes, more preferably 15 minutes, more preferably 30 minutes, such as 60 minutes, 90 minutes or 120 minutes. More preferably the systemic concentration is about 0.5-2.0 ng/kg for the period of time. The systemic concentration may be adjusted according to the response observed in the individual treated and may be adjusted to 0.5 ng/kg, 1.0 ng/kg, 1.5 ng/kg, 2.0 ng/kg, 2.5 ng/kg, 3.0 ng/kg, 3.5 ng/kg or 4.0 ng/kg such as by increasing or decreasing the dosage administered every 15 minutes or so. Preferably the dose is 1.0 ng/kg/min.

In another embodiment the administration takes place for up to about 72 hours, such as for 1 hour, 2 hours, 3 hours, 4 hours, 6 hours, 12 hours, 18 hours, 24 hours, 30 hours, 36 hours, 42 hours, 48 hours, 54 hours, 60 hours, 66 hours or 72 hours. In this embodiment it is preferable that the systemic concentration is about 0.5-2.0 ng/kg for the entire period of time. The systemic concentration may be adjusted according to the response observed in the individual treated and may be adjusted to 0.5 ng/kg, 1.0 ng/kg, 1.5 ng/kg, 2.0 ng/kg, 2.5 ng/kg, 3.0 ng/kg, 3.5 ng/kg or 4.0 ng/kg such as by increasing or decreasing the dosage administered every 15 minutes or so. Preferably the dose is 1.0 ng/kg/min. Although some of the compounds normally are known to have adverse effect on bleeding, it has been found that when administered in the low dosages herein then the desired effect on the endothelium is obtained without the adverse effect on bleeding.

The compound may be administered by a one or more bolus injections, and accordingly, the bolus injection may be given once, twice or several times, for instance, in keeping with the dosage administered the bolus injection may be given every 5 min (minutes), such as every 10 min, such as every 15 min, such as every 20 min, such as every 25 min, such as every 30 min, such as every 35 min, such as every 40 min, such as every 45 min, such as every 50 min, such as every 55 min, such as every 60 min such as every 70 min, such as every 80 min, such as every 90 min, such as every 100 min, such as every 110 min such as every 120 min or more, such as every 180 min, every 240 min or more. For example, the bolus dosage may be administered in the appropriate intervals from the time of trauma to the subject and until a treatment facility such as a hospital or other is reached. The administration may thus take place at a pre-hospital setting, at the hospital, during surgery and/or post-operatively. Thus the bolus may be administered at appropriate intervals for up to about 72 hours.

### Pharmaceutical Compositions of the Invention and its Use

The present invention also relates to a pharmaceutical composition comprising one or more compounds capable of modulating/preserving endothelial integrity particularly prostacyclin or a variant or analogue thereof and a pharmaceutically acceptable carrier. Such pharmaceutically acceptable carrier or excipient as well as suitable pharmaceutical formulation methods are well known in the art (see for example Remington's Pharmaceutical Sciences, 18th ed., Mack Publishing Company, Easton, Pa. (1990). In a preferred embodiment the platelet inhibiting/endothelial protecting variants are prepared in a parenteral composition. Such methods for preparing parenterally administrable compositions will also be known or apparent to those skilled in the art and are described in more detail in, for example, Remington's Pharmaceutical Sciences, 18th ed., Mack Publishing Company, Easton, Pa. (1990). As used herein, the term “pharmaceutical acceptable” means carriers or excipients that does not cause any untoward effects in subjects to whom it is administered.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compositions for parenteral administration comprise the compound as defined above, preferably dissolved in, a pharmaceutically acceptable carrier, preferably an aqueous carrier. A variety of aqueous carriers may be used, such as water, buffered water, saline e.g. such as 0.7%, 0.8%, 0.9% or 1%, glycine such as 0.2%, 0.3%, 0.4% or 0.5% and the like. Normally, it is aimed that the composition has an osmotic pressure corresponding to a 0.9% w/w sodium chloride solution in water. Moreover, as known by a person skilled in the art, dependent on the specific administration route, pH may be adjusted within suitable ranges centred around pH 7.4. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration.

The parenteral formulations typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Following trauma, a pre-prepared formulation may be of a compound as described above in a form that allows immediate administration i.e. in a pre-prepared syringe (for i.e. intra muscular, intravenous, intraosseuos or subcutaneous administration) or tablet or other mucosal application form. This formulation may be administered to the subject at the scene, in an ambulance or helicopter, ie. in a pre-hospital setting.

An embodiment of the invention thus relates to a pre-prepared syringe with a content befitting the average adult or child human being. The average adult or child human weight after which the amount of a compound is calculated may be adapted to suit specific circumstances such as children of different age groups (they are expected to increase in weight with age) or different nationalities, as different nations have different mean weights of their inhabitants. Likewise, a pre-prepared syringe may be made for the specific purpose of having a duration of 5 min, 10 min, 15 min, 30 min, or 60 min or anything therein between.

Thus, the compound as defined above may be formulated so it can be stored at room temperature in preformed bags or syringes containing the solution with the compound capable of modulating/preserving endothelial integrity particularly prostacyclin or a variant or analogue thereof. The concentration of the compound is predefined enabling immediate dosing based on the patients weight regardless of age and gender. The preformed bag may be a 1 liter or a 500 ml or any other conventionally sized bag formulated to tolerate light and be stable at room temperature. The syringe may be a 50 ml syringe, or a syringe of any conventional size such as between 10 ml and 100 ml.

The compositions may contain pharmaceutically acceptable auxiliary substances as required to approximate physiological conditions, such as pH adjusting and buffering agents, stabilizing agents, preservatives, non-ionic surfactants or detergents, antioxidants, tonicity adjusting agents and the like, for example, sodium acetate, sodium lactate, sodium chloride, potassium chloride, calcium chloride, etc.

The compounds of the present invention may also be formulated for sublingual administration. Sublingual administration is particularly suitable for administration to patients with swallowing difficulties, for paediatric use or trauma patients. Patients may have difficulty in swallowing because of a throat disorder or injury and the presently claimed formulation is particularly beneficial in these cases. Patients may also not have a large quantity of saliva so that a larger tablet may not be completely and rapidly dissolved if at all. Passage of an un-dissolved dosage form from the mouth into the throat is thus undesirable and is avoided using the formulations of the invention. It is therefore to minimise the size of the dosage form and dosage forms in accordance with this invention preferably have a minimum size, eg 6 mm diameter and corresponding weight whilst maintaining the dosage. Preferably the total tablet weight does not exceed 100 mg, and more preferably it is less than 70 mg. Rapid dissolution of the dosage form which is necessary to facilitate sublingual absorption may be achieved by selection of an appropriate method of tablet manufacture. Use of direct compression or dry granulation has been found to be less suitable than wet granulation, due to the high bulk density and electrostatic properties of morphine salts, for example morphine sulphate, and excipients.

A specially preferred embodiment of this aspect of the present invention comprises a pre-prepared formulation of compound as defined above that may be stored at ambient temperature, i.e. room temperature, and which also is unaltered (i.e. the compounds do not degrade/breakdown become metabolized or otherwise loose their activity) if exposed to light. Furthermore it is preferred if the formulation is such that it may be administered in the correct dosage immediately.

### Clinical Indications

As described herein above the present invention relates to treatment and/or prevention of acute traumatic coagulopathy (ATC) and prevention of the sequelae following resuscitated cardiac arrest.

### Acute Traumatic Coagulopathy (ATC)

In trauma, physiological compensation mechanisms are initiated with the initial peripheral mesenteric vasoconstriction to shunt blood to the central circulation. If circulation is not restored, hypovolaemic shock ensures (multiple organ failure due to inadequate perfusion.) Trauma patients may develop hypothermia due to environmental conditions at the scene, inadequate protection, intravenous fluid and blood product administration and ongoing blood loss. Deficiencies in coagulation factors and platelets can result from blood loss, dilution, consumption or transfusions. Meanwhile, acidosis and hypothermia interfere with normal blood clotting mechanisms. Thus, coagulopathy develops which may mask surgical bleeding sites and hamper control of mechanical bleeding. Hypothermia, coagulopathy and acidosis are often characterized as the “lethal triad” as these conditions often lead to uncontrollable blood loss, multiple organ failure and death typically in an intensive care unit.

Acute traumatic coagulopathy (ATC) may be defined as an impairment of hemostasis that may occur early after injury and is associated with a four-fold higher mortality, increased transfusion requirements and worse organ failure. ATC appears to have an endogenous component due to the combined shock and tissue damage (trauma) and the absence of exogenous factors such as hemodilution or hypothermia. It has also been suggested that injury severity is positively associated with the development of ATC and hemorrhagic shock has also been implicated. A recent study by Frith et al. showed that the severity of ATC correlated strongly with the combined degree of injury and shock [Frith et al., 2010].

There is however also a need for identifying patients at risk of developing or having developed ATC at the site of injury, i.e. pre-hospital. Patients at risk of developing or suffering from ATC may be identified as described below.

### Traumas

One general aspect of the invention relates to methods of treatment of ATC patients suffering from various forms of trauma, in particularly trauma that may lead to shock as defined above. The trauma may be any type of trauma such as blunt trauma and penetrating trauma; the invention is particularly well suited for treating bleeding following penetrating trauma.

The trauma may be towards the head and/or neck including but not limited to the brain, eye(s), ear(s), nose, mouth, esophagus, trachea, soft tissues, muscles, bones and/or vessel(s) in a subject and/or trauma towards the thoracic region including but not limited to the heart, lungs, oesophagus, soft tissues, muscles or any vessel or vessels in a subject.

Furthermore, the trauma may be towards the abdomen, including but not limited to the liver, pancreas, spleen, ventricle, gall-bladder, intestines, or retroperitoneal tissue, soft tissues, muscles or any vessel or vessels in a subject, and/or towards the pelvis including but not limited to prostate, urinary bladder, uterus, ovarii, bones i.e. pelvic ring, hip, femur, soft tissues, muscles or any vessel or vessels in a subject.

Also, the trauma may be towards the long bones of the extremities including but not limited to humerus, ulnae, radii and/or bones of the hand, femur, tibia, fibula and/or bones of the foot, the columnae, scapulae, costae, clavicle or in any combination hereof in a subject.

### Cardiac Arrest

The inventors have also found that cardiac arrest, (also known as cardiopulmonary arrest or circulatory arrest) leads to severe endothelial dysfunction as defined above. Cardiac arrest is the cessation of normal circulation of the blood due to failure of the heart to contract effectively and if this is unexpected, can be termed a sudden cardiac arrest or SCA.

Arrested blood circulation prevents delivery of oxygen to the body. Lack of oxygen to the brain causes loss of consciousness, which then results in abnormal or absent breathing. Brain injury is likely if cardiac arrest goes untreated for more than five minutes. For the best chance of survival and neurological recovery, immediate and decisive treatment is imperative.

A particular embodiment of the invention relates to a method of treating patients that have been resuscitated from cardiac arrest comprising immediately administering one or more compounds capable of modulating/preserving the endothelial integrity as defined above, such as but not limited to prostacyclin.

Identification of Patients at Increased Risk of Development of ATC by Determination of Syndecan-1, B-Glucose, B-Lactate, and/or APTT Values

It is preferred that the identification of the patients may be performed at an early stage, preferably at the site of the trauma or injury, whereby the treatment may be initiated immediately.

Therefore, a first embodiment of a first aspect of the invention relates to a method of diagnosing, monitoring or determining the likelihood of developing Acute Traumatic

Coagulopathy, such as pre-hospital, wherein said method is capable of identifying a patient who has a significantly increased risk of developing Acute Traumatic Coagulopathy, said method comprising the steps of
a) determining and/or measuring the concentration of at least one of Syndecan-1, sCD44, B-glucose, B-lactate, BE or APTT in a whole blood sample from the patient,
b) comparing said concentration with a predetermined cutoff value, wherein said cutoff value is
   i) Syndecan-1 2-fold higher than normal and/or
   ii) B-glucose 50% higher than normal and/or
   iii) B-lactate 3.5 fold higher than normal and/or
   iv) APTT above normal,
c) wherein a Syndecan-1 value higher than the cutoff value and/or a B-glucose value higher than the cutoff value and/or a B-lactate value higher than the cutoff and/or a APTT value higher than the cutoff value is indicative of a significantly increased risk of developing Acute Traumatic Coagulopathy.

### Syndecan-1

Syndecan is a transmembrane (type I) heparan sulfate proteoglycan and is a member of the syndecan proteoglycan family. The syndecans mediate cell binding, cell signaling, and cytoskeletal organization and syndecan receptors are required for internalization of the HIV-1 tat protein. Syndecan functions as an integral membrane protein and participates in cell proliferation, cell migration and cell-matrix interactions via its receptor for extracellular matrix proteins. Syndecan-1 is also denoted CD138.

Syndecan-1 may be detected using conventional ELISA methods, such as the Human Syndecan-1/CD138 ELISA Kit from CellSciences.

Syndecan-1 may also be detected using lateral flow assays (sticks) similar to those used in e.g. pregnancy tests.

Determination of Syndecan-1 is particularly relevant when the diagnosis is to be established at the place of trauma to initiate the treatment before the patient enters the hospital.

Accordingly, the present invention also relates to a kit for diagnosing, monitoring or determining the likelihood of developing ATC, comprising means for determining Syndecan-1, optionally in combination with means for determining blood-glucose, and/or such as a portable kit that is suitable for pre-hospital use.

In particular the patient has developed or is at risk of development of ATC if the concentration of Syndecan-1 is above a cutoff value, wherein said cutoff value is 2 fold higher than normal. In plasma the cutoff value is at least 50 ng/ml, such as at least 60 ng/ml, more preferably at least 70 ng/ml (in plasma).

### B-Glucose

Measurement of B-glucose may also aid in determination of the risk of development of ATC. If B-glucose is higher than a cutoff which is 50% of the normal value, then it is indicative of an increased risk of developing ATC. This cut-off value in plasma is 7.5 mmol/I.

### B-Lactate

Measurement of B-lactate may also aid in determination of the risk of development of ATC. If B-lactate is higher than a cutoff which is 3.5 fold of the normal value, then it is indicative of an increased risk of developing ATC. This cut-off value in plasma is 3.5 mmol/l.

### APTT

Measurement of APTT may also aid in determination of the risk of development of ATC. If APTT is higher than a cutoff which is just above normal, then it is indicative of an increased risk of developing ATC. The normal value in plasma is 35 seconds.

Other markers include, but are not limited to Base Excess and sCD44.

### Identification of Patients at Increased Risk of Development of ATC by Viscoelastical Citrated Whole Blood Haemostasis Assay: Thrombelastography (TEG) or Thrombelastometry (ROTEM)

If the identification of patients at risk of acquiring ATC is carried out at the hospital or the like one or more of the following diagnostic tests may be used as well.

The TEG in vitro assay is suitable for determining important parameters in the clotting activity and clot strength. The TEG system's approach to monitoring patient haemostasis is based on the premise that the end result of the haemostatic process is the clot. The clot's physical properties determine whether the patient will have normal hemostasis, or will be at increased risk for haemorrhage or thrombosis [Salooja et al. 2001].

The TEG analyzer uses a small whole blood sample in a rotating cup and a pin suspended in the blood by a torsion wire, which is monitored for motion. To speed up the clot formation, a standardized amount of an activator of coagulation (e.g. Kaolin, tissue factor) may be added to the cup just before the pin is placed in the cup. The torque of the rotating cup is transmitted to the immersed pin only after fibrin and/or fibrin-platelet bonding has linked the cup and pin together. The strength and rate of these bonds affect the magnitude of the pin motion such that strong clots move the pin directly in phase with cup motion. Thus, the TEG technology documents the interaction of platelets with the protein coagulation cascade from the time of placing the blood in the analyzer until initial fibrin formation, clot rate strengthening and fibrin-platelet bonding via GPIIb/IIIa, through eventual clot lysis. The TEG R parameter reflects the initiation phase, reaction time, from start of coagulation until the first fibrin band is formed; the Angle (α) represents the increase in clot strength, clot kinetics, correlating with the thrombin generation. The maximal amplitude (MA) parameter reflects maximal clot strength i.e. the maximal elastic modus of the clot. Ly30 demonstrate the proportion of the clot that is dissolved 30 min after MA is reached, reflecting fibrinolysis.

The clot strength and stability and changes herein may be measured as increases in relative clot strength by the TEG (Thrombelastography) measurable parameter MA and clot stability by the TEG derivable parameter Lysis AUC. The maximal amplitude (MA) parameter reflects maximal clot strength i.e. the maximal elastic modus of the clot. The area under the lysis curve, i.e. area under the curve from MA is obtained (Lysis AUC) reflects degree of fibrinolysis. Both clot strength and stability may be measured, or one parameter only may be followed during a procedure such as either the clot stability or the clot strength. It is an object of the present invention that the clot strength measured by the MA increases relative to the MA prior to administration of a compound capable of modulating/preserving endothelial integrity particularly prostacyclin or a variant or analogue thereof by 105%, such as by 110%, such as by 115%, such as by 120%, such as by 125%, such as by 130%, such as by 135%, such as by 140%, such as by 145%, such as by 150%, such as by 155%, such as by 160%, such as by 165%, such as by 170%, such as by 175%, such as by 180%, such as by 185%, such as by 190%, such as by 195%, such as by 200% or more. Likewise it is an object of the present invention that the clot stability increases Lysis AUC. This parameter may with a TEG analysis be measured e.g. after addition of tissue plasminogen activator (tPA), and thus it is an object of the present invention that the clot stability measured by the Lysis AUC increases relative to the Lysis AUC prior to administration of a sympathicomimetic agonist by 105%, such as by 110%, such as by 115%, such as by 120%, such as by 125%, such as by 130%, such as by 135%, such as by 140%, such as by 145%, such as by 150%, such as by 155%, such as by 160%, such as by 165%, such as by 170%, such as by 175%, such as by 180%, such as by 185%, such as by 190%, such as by 195%, such as by 200% or more.

The TEG system has been recognized as a uniquely useful tool and has been used extensively in the management of haemostasis during major surgical interventions such as liver transplantations [Kang et al 1985] and cardiovascular procedures as well as obstetrics, trauma, neurosurgery, management of deep vein thrombosis, and the monitoring and differentiation among platelet GPIIb/IIIa antagonists [Di Benedetto 2003]. TEG-guided transfusion therapy aiming at normalising clot strength (MA) has resulted in a reduction in the use of blood products, a reduction in the rate of re-exploration, prediction of bleeding in cardiac surgery. It has also been employed in the monitoring of heart assist devices. The clinical utility of the TEG comes from that this analysis identifies and quantifies the patient's ability to generate thrombin and the resulting physical properties of the clot as well as identifying enhanced fibrinolysis [Rivard et al. 2005].

In one embodiment, the invention thus relates to a method of identifying patients at increased risk of developing ATC by analyzing a citrated whole blood sample, such as in a citrated whole blood sample activated by kaolin, such as in a citrated whole blood sample activated by tissue factor, such as in a native whole blood sample, such as a native whole blood sample activated by kaolin, such as in a citrated whole blood sample activated by tissue factor from the patient by a cell based viscoelastical assay upon arrival at the ICU.

In one embodiment, the invention thus relates to a method of identifying patients at increased risk of developing ATC by analyzing a citrated whole blood sample from the patient by the thrombelastography (TEG) system.

In one embodiment, the invention thus relates to a method of identifying patients at increased risk of developing ATC by analyzing a citrated whole blood sample from the patient by the thrombelastometry (ROTEM) systems.

Thus a particular embodiment relates to a method of diagnosing, monitoring or determining the likelihood of developing Acute Traumatic Coagulopathy, wherein said method is capable of identifying patients who have a significantly increased risk of developing Acute Traumatic Coagulopathy, said method comprising the steps of
i) determining/measuring at least one of the blood coagulation parameters APTT, PT and PTr,
ii) comparing said value with a predetermined cutoff value, wherein said cutoff value is
   a) APTT higher than 35 seconds, such as higher than 35 seconds,
   b) PT higher than 1.1, such as higher than 1.2,
   c) PTr higher than 1.1, such as higher than 1.2.

Another particular embodiment relates to a method of diagnosing, monitoring or determining the likelihood of developing Acute Traumatic Coagulopathy, wherein said method is capable of identifying patients who have a significantly increased risk of developing Acute Traumatic Coagulopathy, said method comprising the steps of
i) Determining/measuring at least one of the viscoelastical data points R, Angle and MA by thromboelastography (TEG) in a whole blood sample from the patient, such as in a citrated whole blood sample, such as in a citrated whole blood sample activated by kaolin,
ii) comparing said concentration with a predetermined cutoff value, said cutoff value being an equivalent to a cutoff value determined by TEG in a citrated whole blood sample activated by kaolin wherein said cutoff value is
   a) R higher than 8.0 minutes, such as higher than 11 minutes, such as higher than 12 minutes,
   b) Angle lower than 60°, such as lower than 55°,
   c) MA lower than 51 mm, such as lower than 50 mm,
   d) Ly30 higher than 7% such as higher than 8%,
      wherein an R-value higher than the cutoff value and/or an Angle-value lower than the cutoff value and/or a MA lower than the cutoff value and/or a Ly30 value higher than the cutoff value is indicative of a significantly increased risk of developing Acute Traumatic Coagulopathy as compared to a human being wherein neither R or Ly30 are higher or Angle-value or MA are lower than the cutoff value.

Yet another particular embodiment relates to a method of diagnosing, monitoring or determining the likelihood of developing Acute Traumatic Coagulopathy, wherein said method is capable of identifying patients who have a significantly increased risk of developing Acute Traumatic Coagulopathy, said method comprising the steps of
i) Determining/measuring at least one of the viscoelastical data points Clotting time. Clot formation time, Angle, CA5 and MCF by thromboelastometry (ROTEM) in a whole blood sample from the patient, such as in a citrated whole blood sample, such as in a citrated whole blood sample activated by kaolin,
ii) comparing said concentration with a predetermined cutoff value, said cutoff value being an equivalent to a cutoff value determined by TEG in a citrated whole blood sample activated by kaolin wherein said cutoff value is
   a) Clotting time higher than 65 seconds, such as higher than 70 seconds and/or
   b) Clot formation time higher than 110 seconds, such as higher than 120 seconds and/or
   c) Angle lower than 75 degrees, such as lower than 70 degrees and/or
   d) CA5 lower than 45 mm, such as lower than 40 mm and/or
   e) MCF lower than 60 mm, such as lower than 55 mm,
      wherein a clotting time higher than the cutoff value and/or a clot formation time higher than the cutoff value, an Angle-value lower than the cutoff value and/or a CA5 value lower than the cutoff value and/or a MCF lower than the cutoff value is indicative of a significantly increased risk of developing Acute Traumatic Coagulopathy as compared to a human being wherein neither clotting time or clot formation time are higher than the cutoff value or Angle, CA5 or MCF values are lower than the cutoff value.

### Kit of Parts

Further embodiments of the invention relate to kits of parts.

A particular embodiment relates to a kit for use in the treatment and/or prophylaxis of Acute Traumatic Coagulopathy according to any of the preceding claims, comprising
i) Prostacyclin (or an analogue or variant hereof) alone or in combination with endothelial/modulating compounds as described above,
ii) optionally an aqueous medium to dissolve the compound, and
iii) optionally, instructions for use.

Another embodiment relates to a kit for use in the treatment and/or prophylaxis of the sequelae following resuscitated cardiac arrest according to any of the preceding claims, comprising
i) a prostacyclin alone or in combination with endothelial/modulating compounds as described above,
ii) optionally an aqueous medium to dissolve the compound, and
iii) optionally, instructions for use.

Yet another embodiment relates to a kit wherein the
i) prostacyclin alone or in combination with endothelial/modulating compounds and
ii) optionally an aqueous medium to dissolve the compound, formulated as a pre-prepared formulation for intramuscular, intravenous or subcutaneous administration, such as a pre-prepared syringe.

It is an option to include in any of the above kits a second active ingredient such as but not limited to adrenergic antagonists as disclosed above.

### REFERENCES

Allard C B, Scarpelini S, Rhind S G et al. Abnormal coagulation tests are associated with progression of traumatic intracranial hemorrhage. J Trauma 2009; 67(5):959-967.
Arbabi S, Campion E M, Hemmila M R et al. Beta-blocker use is associated with improved outcomes in adult trauma patients. J Trauma 2007; 62:56-61
Atkinson et al., Blood Cells, Molecules, and Diseases 36 (2006) 217-222
Bentzer P, Grande P O. Low-dose prostacyclin restores an increased protein permeability after trauma in cat skeletal muscle. J Trauma 2004; 56:385-392
Bentzer P, Mattiasson G, McIntosh T K et al. Infusion of prostacyclin following experimental brain injury in the rat reduces cortical lesion volume. J Neurotrauma 2001; 18:275-285
Bentzer P, Venturoli D, Carlsson 0 et al. Low-dose prostacyclin improves cortical perfusion following experimental brain injury in the rat. J Neurotrauma 2003; 20:447-461
Bihari et al., Intensive Care Med. 1988; 15(1):2-7
Bitterman H, Lefer D J, Lefer A M. Novel mechanism of action of a prostacyclin enhancing agent in haemorrhagic shock. Naunyn Schmiedebergs Arch Pharmacol 1988a; 337:679-686
Bitterman H, Smith B A, Lefer D J et al. Salutary effects of CG-4203, a novel, stable prostacyclin analog, in hemorrhagic shock. J Cardiovasc Pharmacol 1988b; 12:293-299
Bitterman H, Stahl G L, Lefer A M. Protective effects of CG-4203, a novel stable prostacyclin analog, in traumatic shock. Prostaglandins 1988c; 35:41-50
Brohi K, Cohen M J, Ganter M T et al. Acute traumatic coagulopathy: initiated by hypoperfusion: modulated through the protein C pathway? Ann Surg 2007; 245:812-818
Brohi K, Cohen M J, Ganter M T et al. Acute coagulopathy of trauma: hypoperfusion induces systemic anticoagulation and hyperfibrinolysis. J Trauma 2008; 64:1211-1217
Brohi K, Singh J, Heron M et al. Acute traumatic coagulopathy. J Trauma 2003; 54:1127-1130
Cines et al., 1998, Blood 91:3527-3561.
Cohen M J, Serkova N J, Wiener-Kronish J et al. 1H-NMR-based metabolic signatures of clinical outcomes in trauma patients—beyond lactate and base deficit. J Trauma 2010; 69:31-40
Colgan et al., Purinergic Signalling (2006) 2: 351-360.
Costantini T W, Peterson C Y, Kroll L et al. Burns, inflammation, and intestinal injury: protective effects of an anti-inflammatory resuscitation strategy. J Trauma 2009; 67:1162-1168
Di Benedetto et al., Minerva Anestesiol. 2003 June; 69(6):501-9, 509-15.
Frith D, Brohi K. The acute coagulopathy of trauma shock: Clinical relevance. Surgeon 2010; 8:159-163
Gando S, Kameue T, Matsuda N et al. Combined activation of coagulation and inflammation has an important role in multiple organ dysfunction and poor outcome after severe trauma. Thromb Haemost 2002; 88:943-949
Ganter M T, Brohi K, Cohen M J et al. Role of the alternative pathway in the early complement activation following major trauma. Shock 2007; 28:29-34
Goepfert et al., 2000, Molecular Medicine 6(7): 591-603.
Grande P O, Moller A D, Nordstrom C H et al. Low-dose prostacyclin in treatment of severe brain trauma evaluated with microdialysis and jugular bulb oxygen measurements. Acta Anaesthesiol Scand 2000; 44:886-894
Kang et al., Anesth Analg. 1985 September; 64(9):888-96.
Keel M, Trentz O. Pathophysiology of polytrauma. Injury 2005; 36:691-709
Lefer A M, Araki H. Analysis of potential beneficial actions of prostaglandins in traumatic shock. Prog Clin Biol Res 1983; 111:199-210
Lefer A M, Sollott S L, Galvin M J. Beneficial actions of prostacyclin in traumatic shock. Prostaglandins 1979; 17:761-767
Levitt M A, Lefer A M. Anti-shock properties of the prostacyclin analog, iloprost, in traumatic shock. Prostaglandins Leukot Med 1986; 25:175-185
Lundblad C, Grande P O, Bentzer P. Increased cortical cell loss and prolonged hemodynamic depression after traumatic brain injury in mice lacking the IP receptor for prostacyclin. J Cereb Blood Flow Metab 2008; 28:367-376
MacLeod J B, Lynn M, McKenney M G et al. Early coagulopathy predicts mortality in trauma. J Trauma 2003; 55:39-44
Maegele M, Lefering R, Yucel N et al. Early coagulopathy in multiple injury: an analysis from the German Trauma Registry on 8724 patients. Injury 2007; 38:298-304
Malone D L, Dunne J, Tracy J K et al. Blood transfusion, independent of shock severity, is associated with worse outcome in trauma. J Trauma 2003; 54:898-905
Maier B, Lefering R, Lehnert M et al. Early versus late onset of multiple organ failure is associated with differing patterns of plasma cytokine biomarker expression and outcome after severe trauma. Shock 2007; 28:668-674
Marsolais et al., Nat Rev Drug Discov. 2009 April; 8(4):297-307.
Moore F A, Sauaia A, Moore E E et al. Postinjury multiple organ failure: a bimodal phenomenon. J Trauma 1996; 40:501-510
Naredi S, Olivecrona M, Lindgren C et al. An outcome study of severe traumatic head injury using the “Lund therapy” with low-dose prostacyclin. Acta Anaesthesiol Scand 2001; 45:402-406
Peden, M., McGee, K., and Krug, E. Injury: A leading cause of the global burden of disease, 2000. Peden, M., McGee, K., and Krug, E. Available at: http://whqlibdoc.who.int/publications/2002-9241562323.pdf. 2002. Geneva, Switzerland, World Health Organization.
Rivard et al., 2005, Journal of Thrombosis and Haemostasis, 4: 411-416
Rough J, Engdahl R, Opperman K et al. beta2 Adrenoreceptor blockade attenuates the hyperinflammatory response induced by traumatic injury. Surgery 2009; 145:235-242
Sahsivar M O, Narin C, Kiyici A et al. The effect of iloprost on renal dysfunction after renal I/R using cystatin C and beta2-microglobulin monitoring. Shock 2009; 32:498-502
Salooja et al., Blood Coagul Fibrinolysis. 2001 July; 12(5):327-37. Review. Erratum in: Blood Coagul Fibrinolysis 2002 January; 13(1):75.
Sauaia A, Moore F A, Moore E E et al. Early predictors of postinjury multiple organ failure. Arch Surg 1994; 129:39-45
Sauaia A, Moore F A, Moore E E et al. Epidemiology of trauma deaths: a reassessment. J Trauma 1995; 38:185-193
Schereen et al., Intensive Care Med (1997) 23: 146-158
Stahel P F, Smith W R, Moore E E. Role of biological modifiers regulating the immune response after trauma. Injury 2007; 38:1409-1422
Starling M B, Neutze J M, Hill D G et al. The effects of prostacyclin (PGI2) on haematological and haemodynamic parameters, and lung histology in puppies undergoing cardiopulmonary bypass surgery with profound hypothermia. Prostaglandins Leukot Med 1985; 17:11-29
Stein S C, Young G S, Talucci R C, Greenbaum B H, Ross S E. Delayed brain injury after head trauma: significance of coagulopathy. Neurosurgery 1992; 30(2):160-165.
Tamura M. Protective effects of a PGI2 analogue OP-2507 on hemorrhagic shock in rats—with an evaluation of the metabolic recovery using near-infrared optical monitoring. Jpn Circ J 1992; 56:366-375
Thompson et al., J. Exp. Med. Volume 200, Number 11, December 6,2004 1395-1405
Wafaisade A, Wutzler S, Lefering R et al. Drivers of acute coagulopathy after severe trauma: a multivariate analysis of 1987 patients. Emerg Med J 2010
Xu et al., Nat Med. 2009 November; 15(11):1318-21.
Zardi et al., International Immunopharmacology 5 (2005) 437-459
Zardi et al., Prostaglandins & other Lipid Mediators 83 (2007) 1-24

### EXAMPLES

### Example 1

### Safety Using Prostacyclin in Bleeding Patients

Ninety-four critically ill patients admitted to the intensive care unit (ICU) underwent haemofiltration with or without concomitant Flolan (prostacycline) treatment. None of the patients were suffering from Acute Traumatic Coagulopathy nor from sequelae to cardiac arrest. Flolan was administered in a low dose in the filters to prevent these from clotting and consequently there was only a minor spill over of Flolan to the systemic circulation. The patients were retrospectively reviewed.

TABLE 6
Demography of ICU patients

| | Flolan group | Non-Flolan group |
|---|---|---|
| | (n = 24) | (n = 70) |
| APACHE II score (mean) | 26 | 28 |
| Platelet count (difference before | +14 | −17 |
| vs. after haemofiltration) | | |
| 90 day mortality (%) | 34 | 53 |

APACHE II: Acute Physiology and Chronic Health Evaluation II,
ICU: Intensive Care Unit

The two groups (Flolan vs non-flolan) were comparable with regards to APACHE II at admission. However, patients in the flolan group were more severely ill as evaluated by a lower platelet count at start of hemofiltration, a higher frequency of severe thrombocytopenia, a higher frequency of DIC diagnoses, a higher maximum SOFA score and a higher SOFA score at hemofiltration initiation as compared to the patients receiving non-flolan. The finding of increased total transfusion requirements and specifically of FFP (Fresh Frozen Plasma) during hemofiltration in the flolan group vs. the non-flolan group might thus be attributed to the higher disease severity and associated coagulopathy and not to an increased risk of bleeding due to the use of flolan as anticoagulant.

Importantly, when comparing mortality between groups, we found that the flolan group tended to have decreased mortality at 30 days (21% vs. 39%, p=0.12), 90 days (34% vs. 53%, p=0.10) and 365 days (38% vs. 57%, p=0.09).

Flolan, in the dosages administered, does not negatively influence the haemostatic competence as evaluated by transfusion requirements in critically ill patients undergoing haemofiltration and thereby questions the assumption that prostacycline is a powerful antithrombotic agent.

Furthermore, the significant decrease in mortality observed in haemofiltrated patients receiving flolan in the filters indicates that the minor systemic spill-over affects the endothelium beneficially by limiting the pro-coagulant effects of systemic inflammation and coagulation activation and thereby preventing microvascular occlusion and organ failure.

### Example 2

### Safety of Treatment in Healthy Volunteers

Six healthy volunteers were administered flolan (Prostacycline) intravenously at a dose of 4 ng/kg/min for 2 h. Blood samples for whole blood viscoelastical assay (Thrombelastography [TEG]) and whole blood platelet aggregation (Multiplate) were obtained before infusion of Flolan, after 60 min infusion of Flolan and after 120 min infusion of Flolan.

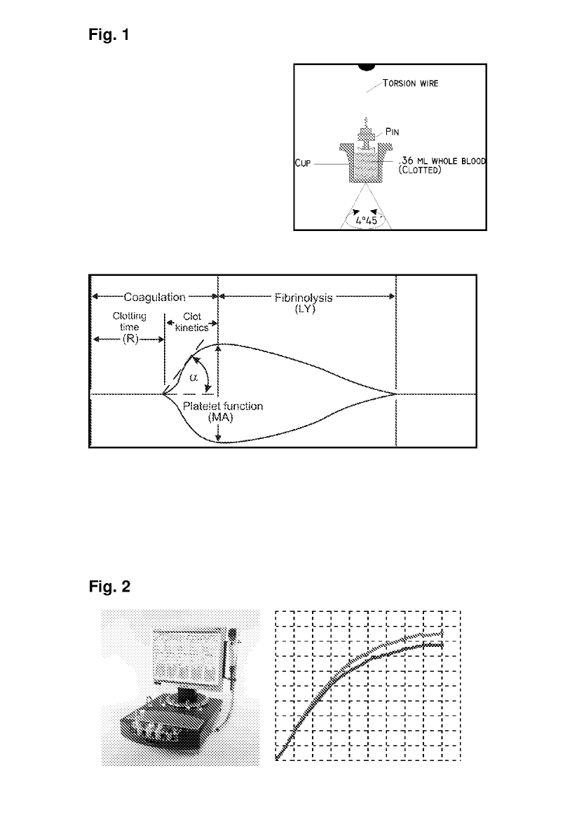

With regard to the TEG assay this was performed as recommended by the manufacturer and 340 μl are mixed with 20 μl CaCl 0.2 M (final concentration 11.1 mM in the cup) and kaolin at 37° C. after which the haemostatic activity is recorded as depicted in FIG. 1.

Whole blood impedance aggregometry was analyzed by the Multiple Platelet function Analyzer (MultiPlate® analyzer). Analysis employing various platelet agonists: ASPItest (activation by arachidonic acid), COLtest (activation by collagen through the collagen receptor), TRAPtest (activation by TRAP-6 stimulates the thrombin receptor on the platelet surface and ADPtest (activation by ADP stimulates platelet activation by the ADP receptors).

MultiPlate continuously records platelet aggregation. The increase of impedance by the attachment of platelets onto the Multiplate sensors is transformed to arbitrary aggregation units (AU) and plotted against time as depicted in FIG. 2.

### Results:

Prostacyclin in the doses administered did not change blood pressure or heart rate from baseline values at any time point during the study period.

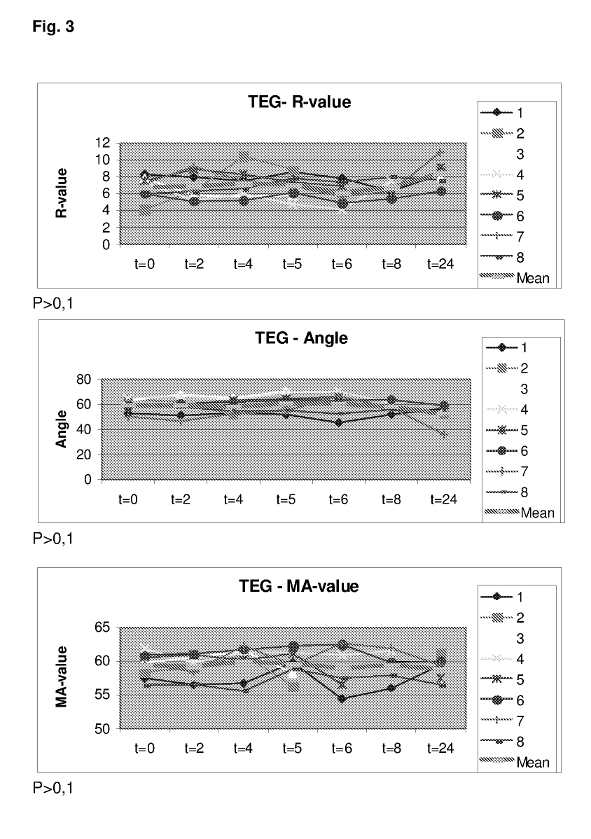

No significant difference was observed when comparing baseline TEG values with samples obtained after 60 and 120 min of flolan infusion for any of the parameters investigated (R, Angle, M A) in any of the 6 volunteers studied, FIG. 3.

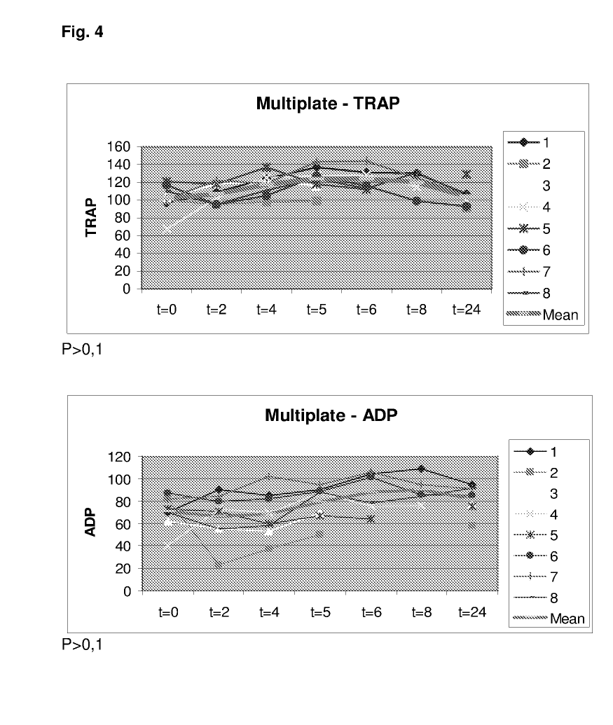

Similarly, no significant difference was observed when comparing baseline Multiplate values with samples obtained after 60 and 120 min of flolan infusion for any of the agonists investigated (ASPI, COL, ADP, TRAP) in any of the 6 volunteers studied, FIG. 4.

### Conclusions:

Infusion of Flolan at the doses recommended for clinical use does not negatively affect whole blood haemostatic competence as evaluated by TEG. Furthermore, with regard to whole blood platelet aggregation employing various platelet agonists is not affected negatively by flolan infusion indicating that such administration does not compromise haemostasis.

### Example 3

### Endothelial Protective and Anticoagulation Effects of Flolan® Infusion in Healthy Subjects

### Study Protocol

Eight healthy volunteers were administered Flolan® (Prostacyclin) intravenously at a dose of 4 ng/kg/min for 2 h. Blood samples were analyzed for plasma biomarkers indicative of endothelial cell (thrombomodulin, PAI-1) and glycocalyx (syndecan-1) activation and/or damage, cellular necrosis (histone-complexed DNA fragments, HMGB1) and anticoagulation (protein C, antithrombin, TFPI) at the following time points: Before the infusion (0 h), immediately after ceasing the infusion (2 h) and then 4 h, 5 h, 6 h, 8 h and 24 h after starting the infusion. The concentration of the individual biomarkers in plasma was analyzed by commercially available ELISA kits according to the manufactures recommendations. Paired t-tests with p-values <0.05 were considered significant.

### Results

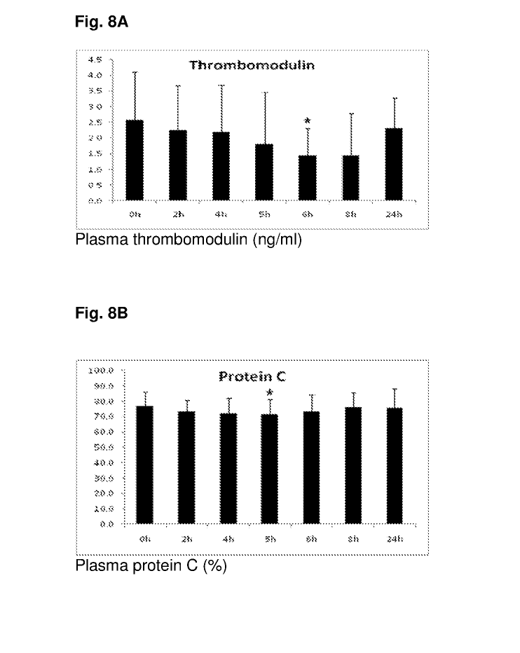

Prostacyclin in the administered dose had an endothelial protective effect evidenced by a marked decrease in the circulating level of thrombomodulin, an effect that seemed to be prolonged and continuing for several hours after ceasing the infusion (FIG. 8A). Furthermore, the circulating level of Protein C decreased in the hours after ceasing the Flolan infusion, indicating that prostacyclin enhanced activation of Protein C (resulting in a decline in the non-activated form of protein C) (FIG. 8B).

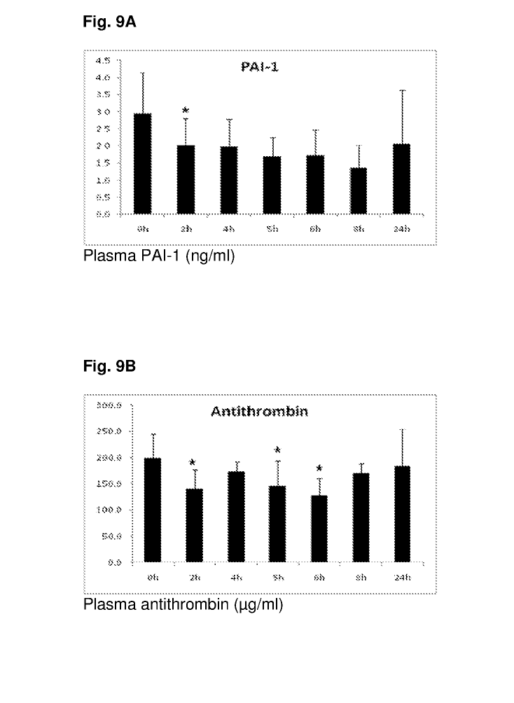

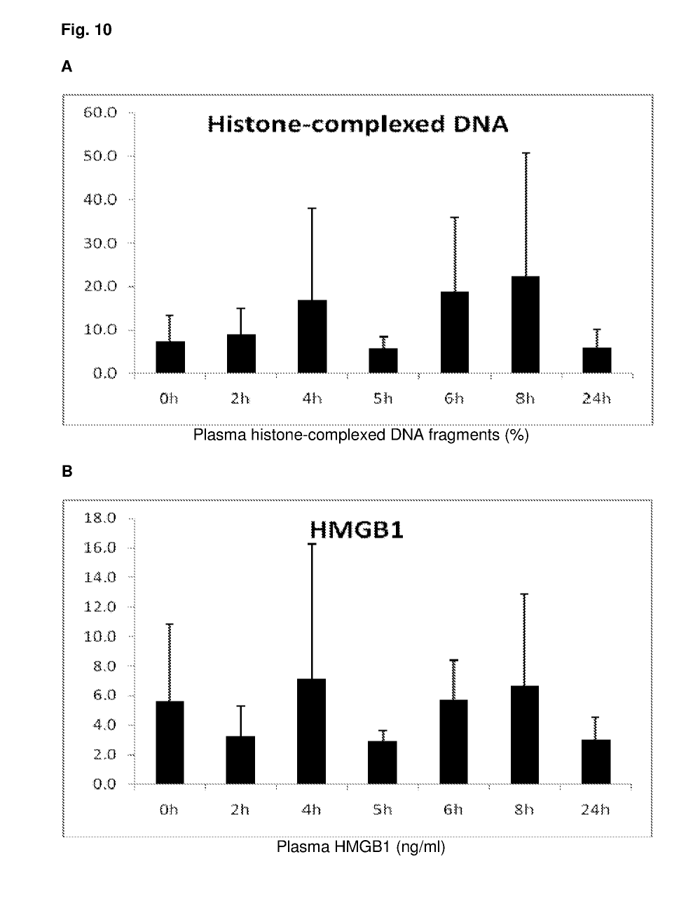

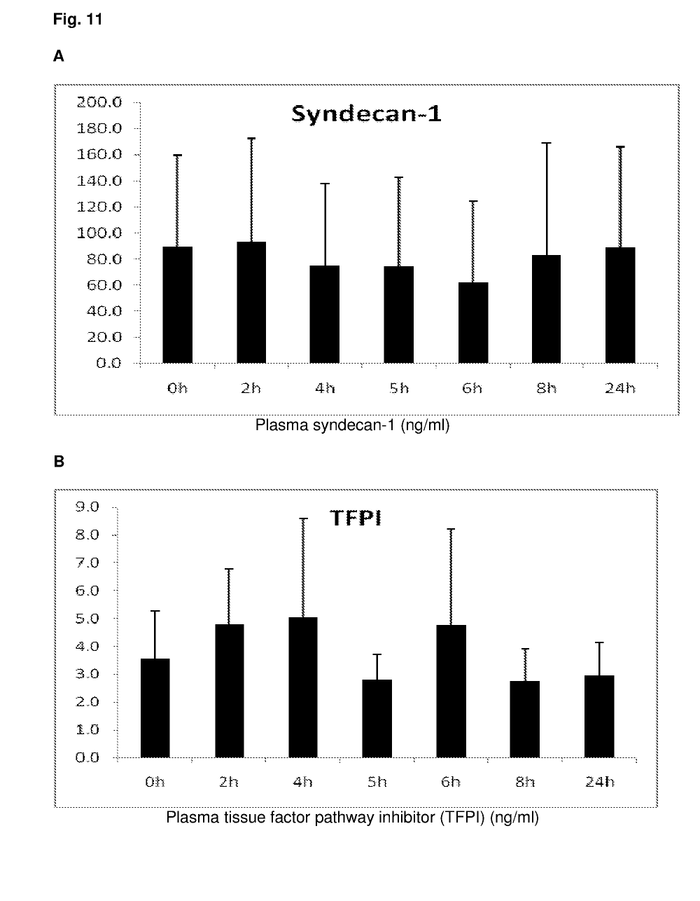

Furthermore, the circulating level of PAI-1, an inhibitor of fibrinolysis shed from the activated endothelium, also declined (FIG. 9A), further indicating that the prostacyclin infusion deactivated the endothelium and enhanced endogenous fibrinolysis. Finally, the circulating level of antithrombin also decreased (FIG. 9B) indicating that a higher amount of this was attached to the endothelial glycocalyx rather than being on a soluble form (FIG. 9B).

Prostacyclin did not influence the cell necrosis biomarkers (histone-complexed DNA fragments, HMGB1) significantly, though there was a tendency towards a reduction in HMGB1 at 5 h (p=0.056) (FIG. 5A-B). Prostacyclin did not induce or influence glycocalyx shedding (syndecan-1) (FIG. 6A) or affected the circulating level of TFPI (FIG. 6B).

### Conclusion

The finding that the administered dose of prostacyclin was associated with concurrent decreases in thrombomodulin and Protein C in healthy individuals is a proof-of-concept of the endothelial protective effect of prostacyclin. Mechanistically, the finding indicates that prostacyclin reduces endothelial release/shedding of thrombomodulin, a recognized marker of endothelial damage, and thereby also increases the amount of protein C that can be activated by/at the endothelium. Activated Protein C exerts a cytoprotective effect on the endothelium through the PAR receptors and high levels of thrombomodulin indicate crude endothelial cell damage and predict high mortality in trauma patients. Given this, this finding identifies for the first time an important mechanism by which prostacyclin may improve outcome in trauma patients as well as patients undergoing major surgery with a high risk of development of capillary leakage syndrome secondary to endothelial modulation. The finding that PAI-1 decreased along with antithrombin during prostacyclin infusion further indicates that prostacyclin both supports fibrinolysis and exerts endothelial protection by increasing antithrombin adhesion to the endothelial glycocalyx.

### Example 4

Patients suffering from acute traumatic coagulopathy (ATC) are administered Iloprost (Prostacyclin) intravenously at a dose of 1 ng/kg/min for 24 h. Blood samples are analyzed for plasma biomarkers indicative of endothelial cell (thrombomodulin, PAI-1) and glycocalyx (syndecan-1) activation and/or damage, cellular necrosis (histone-complexed DNA fragments, HMGB1) and anticoagulation (protein C, antithrombin, TFPI) at the following time points: Before the infusion (0 h), immediately after ceasing the infusion (24 h) and then 4 h, 6 h, 8 h, 12 h, 16 h, 20 h, 24 h, 30 h, 36 h, 48 h, 60 h and 72 h after starting the infusion. The concentration of the individual biomarkers in plasma is analyzed by commercially available ELISA kits according to the manufactures recommendations. Finally, the finding that prostacyclin did not significantly influence the cell damage biomarkers histone-complexed DNA fragments and HMGB1 (though HMGB1 was reduced non-significantly) and the glycocalyx degradation biomarker (syndecan-1) is expected since the healthy subjects included in this study did not suffer from tissue damage.

### Example 5

Patients resuscitated from cardiac arrest are administered Iloprost (Prostacyclin) intravenously at a dose of 1 ng/kg/min for 24 h. Blood samples are analyzed for plasma biomarkers indicative of endothelial cell (thrombomodulin, PAI-1) and glycocalyx (syndecan-1) activation and/or damage, cellular necrosis (histone-complexed DNA fragments, HMGB1) and anticoagulation (protein C, antithrombin, TFPI) at the following time points: Before the infusion (0 h), immediately after ceasing the infusion (24 h) and then 4 h, 6 h, 8 h, 12 h, 16 h, 20 h, 24 h, 30 h, 36 h, 48 h, 60 h and 72 h after starting the infusion. The concentration of the individual biomarkers in plasma is analyzed by commercially available ELISA kits according to the manufactures recommendations.

### Example 6

Patients suffering from acute traumatic coagulopathy (ATC) are administered Iloprost (Prostacyclin) intravenously at a dose of 1 ng/kg/min for 24-72 h. Blood samples are analyzed for plasma biomarkers indicative of endothelial cell (thrombomodulin, PAI-1) and glycocalyx (syndecan-1) activation and/or damage, cellular necrosis (histone-complexed DNA fragments, HMGB1) and anticoagulation (protein C, antithrombin, TFPI) at the following time points: Before the infusion (0 h), immediately after ceasing the infusion (72 h) and then 4 h, 6 h, 8 h, 12 h, 16 h, 20 h, 24 h, 30 h, 36 h, 48 h, 60 h, 72 h, 90 h, 108 h, 120 h, 132 h, and 150 h after starting the infusion. The concentration of the individual biomarkers in plasma is analyzed by commercially available ELISA kits according to the manufactures recommendations.

### Example 7

Patients resuscitated from cardiac arrest are administered Iloprost (Prostacyclin) intravenously at a dose of 1 ng/kg/min for 24-72 h. Blood samples are analyzed for plasma biomarkers indicative of endothelial cell (thrombomodulin, PAI-1) and glycocalyx (syndecan-1) activation and/or damage, cellular necrosis (histone-complexed DNA fragments, HMGB1) and anticoagulation (protein C, antithrombin, TFPI) at the following time points: Before the infusion (0 h), immediately after ceasing the infusion (72 h) and then 4 h, 6 h, 8 h, 12 h, 16 h, 20 h, 24 h, 30 h, 36 h, 48 h, 60 h 72 h, 90 h, 108 h, 120 h, 132 h, and 150 h after starting the infusion. The concentration of the individual biomarkers in plasma is analyzed by commercially available ELISA kits according to the manufactures recommendations.

1. A compound capable of modulating/preserving endothelial integrity for use in prevention or treatment of Acute Traumatic Coagulopathy.

2. A compound capable of modulating/preserving endothelial integrity for use in prevention or treatment of the sequelae that follow resuscitated cardiac arrest.

3. The use according to any of claims 1 and 2 wherein the compound is prostacyclin or variants thereof.

4. The use according to claim 3, wherein the prostacyclin variant is selected from the group consisting of beraprost sodium, epoprostenol sodium, iloprost, flolan, sildenafil citrate, treprostinil, pegylated treprostinil, treprostinil diethanolamine and treprostinil sodium, 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyacetic acid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, and 15-deoxy-16[alpha]-hydroxy-16[beta],20-dimethyl-[DELTA]6,6a-6a-carba PGI1, pentoxifylline (1-{5-oxohexyl}-3,7-dimethylxanthine).

5. The use according to any of claims 1 and 2 wherein the compound is iloprost.

6. The use of any of claims any of the preceding claims, wherein the compound capable of modulating/preserving the endothelial integrity has a half time of less than 4 hours (such as Treprostinil), preferably less than 1 hours (such as Beraprost (35-40 min)), more preferably less than ½ hour (such as Iloprost (20-30 min)), preferably less than 5 min (such as Epoprostenol (0.5-3 min)).

7. The use according to any of the preceding claims, wherein the dose of prostacyclin is administered to maintain a systemic concentration in the range of 0.1 to 4.0 ng/kg.

8. The use according to any of the preceding claims, wherein the prostacyclin is administered parenterally.

9. The use according to claim 8, wherein the parenteral administration is intravenous, intraarterial, subcutaneous, intramuscular, intrapulmonary via the alveoli, intracardiac, intradermal, transdermal, transmucosal, intrathecal, intraperitoneal, intraosseous and/or intravesical or by other means whereby an appropriate systemic concentration is obtained.

10. The use according to claim 8, wherein the parenteral administration is subcutaneous, intramuscular, intraosseous and/or intravenous.

11. The use according to any of the preceding claims, wherein the dose of the compound is administered as a single bolus dose or as repeated doses.

12. The use according to any of the preceding claims, wherein the dose of the compound is administered continuously.

13. The use according to any of the preceding claims, formulated for infusion, injection or in a tablet for immediate use.

14. The use according to any of the preceding claims, in a pre-prepared formulation for intramuscular, intravenous or subcutaneous administration in a pre-prepared syringe.

15. A method of treating or preventing Acute Traumatic Coagulopathy comprising administering to subject in need of such a treatment, an effective dose of one or more compounds as defined in claim 1.

16. A method of treating cardiac arrest comprising administering to subject in need of such a treatment, an effective dose of one or more compounds as defined in claim 1.

17. The method according to any of claims 15 and 16, wherein the compound is as defined in any of claims 3-14.

18. The method according to any of claims 15 and 16, wherein the compound capable of modulating/preserving endothelial integrity is administered simultaneously, separately or sequentially with an endothelial modulator and/or an adrenergic receptor modulator.

19. The method according to claim 18, wherein the adrenergic receptor modulator is an adrenergic receptor agonist, such as but not limited to phenylephrine, clonidine, isoproteterenol, dobutamine and/or epinephrine.

20. The method according to claim 18, wherein the adrenergic receptor modulator is an adrenergic receptor antagonist, such as but not limited to alpha and/or beta receptor antagonists and/or betablokkers.

21. A kit for use in the treatment and/or prophylaxis of Acute Traumatic Coagulopathy according to any of the preceding claims, comprising
i) Prostacyclin or a variant thereof as defined in any of claims 3-14,
ii) optionally in combination with at least one other compound,
iii) optionally an aqueous medium to dissolve the compound, and
iv) optionally instructions for use.

22. A kit for use in the treatment of cardiac arrest according to any of the preceding claims, comprising
i) prostacyclin or a variant thereof as defined in any of claims 3-14,
ii) optionally in combination with at least one other compound,
iii) optionally an aqueous medium to dissolve the compound, and
iv) optionally, instructions for use.

23. The kit according to any of claims 21 and 22 wherein the
i) Prostacyclin or prostacyclin variant and
ii) optionally in combination with at least one other compound,
iii) optionally an aqueous medium to dissolve the compound, and are formulated as a pre-prepared formulation for intramuscular, intravenous or subcutaneous administration, such as a pre-prepared syringe.

24. A kit according to any of claims 21-23, further comprising an adrenergic receptor modulator according to claims 19-20.

25. A pharmaceutical composition comprising a compound as defined in any of claims 1-14 for the treatment or prophylaxis of Acute Traumatic Coagulopathy in a subject.

26. A pharmaceutical composition comprising a compound as defined in any of the claims 1-14 for the treatment of sequelae from cardiac arrest in a subject.

27. A method of diagnosing, monitoring or determining the likelihood of developing Acute Traumatic Coagulopathy, wherein said method is capable of identifying patients who have a significantly increased risk of developing Acute Traumatic Coagulopathy, said method comprising the steps of
i) determining the concentration of at least one of Syndecan-1, B-glucose, B-lactate and APTT in a whole blood sample from the patient,
ii) comparing said concentration with a predetermined cutoff value, wherein said cutoff value is
a) Syndecan-1 2 fold higher than normal
b) B-glucose 50% higher than normal
c) B-lactate 3.5 fold higher than normal
d) APTT above normal
wherein a Syndecan-1 value higher than the cutoff value and/or a B-glucose value higher than the cutoff value and/or a B-lactate value higher than the cutoff and/or a APTT value higher than the cutoff value is indicative of a significantly increased risk of developing Acute Traumatic Coagulopathy.
